(19) United States
(12) Patent Application Publication
(10) Pub. No.: US 2015/0044156 A1
(43) Pub. Date: Feb. 12, 2015

(54) HAIR PREPARATIONS COMPRISING HYDROXY-TERMINATED ORGANOPOLYSILOXANE(S) AND THICKENER(S)

(71) Applicant: Henkel AG & Co. KGaA, Duesseldorf (DE)

(72) Inventors: Erik Schulze zur Wiesche, Hamburg (DE); Markus Semrau, Langenfeld (DE)

(21) Appl. No.: 14/521,694

(22) Filed: Oct. 23, 2014

Related U.S. Application Data
(63) Continuation of application No. PCT/EP2013/053876, filed on Feb. 27, 2013.

(30) Foreign Application Priority Data
Apr. 26, 2012 (DE) 10 2012 206 948.8

(51) Int. Cl. A61K 8/898; A61K 8/81; A61Q 5/12

(52) U.S. Cl. CPC A61K 8/898; A61Q 5/12; A61K 8/8147; A61K 8/8158; A61K 2800/594; A61K 2800/48
USPC 424/70.12

(57) ABSTRACT

Cosmetic compositions include at least one thickener selected from associative thickeners; crosslinked homopolymers of acrylic acid; crosslinked copolymers of (meth)acrylic acid and alkyl(C₁₋₆) acrylate; nonionic homopolymers and copolymers including monomers with ethylenically unsaturated bonds of the ester and/or amide type; homopolymers of ammonium acrylate or copolymers of ammonium acrylate and acrylamide; fatty acid amides and the mixtures thereof with at least one hydroxy-terminated organopolysiloxane of formula (I) as described herein.

### CROSS-REFERENCE TO RELATED APPLICATIONS

The present application is a continuation of International Patent Application No. PCT/EP2013/053876, filed Feb. 27, 2013 and entitled “HAIR PREPARATIONS COMPRISING HYDROXY-TERMINATED ORGANOPLOYSILOXANE(S) AND THICKENER(S)”, which claims priority to DE 10 2012 206 948.8, filed Apr. 26, 2012, by which both are hereby incorporated by reference in their entirety.

### FIELD OF THE INVENTION

The present invention generally relates to hair treatment agents that include specific substituted silicone(s) and to the use of these agents for cleaning and/or conditioning hair.

### BACKGROUND OF THE INVENTION

Conditioning agents for keratinic fibers have an influence on the natural structure and properties of the hair. Accordingly, after such treatments, for example the hair's wet and dry combability and its hold and body may be optimized or the hair may be protected from increased splitting. It has therefore long been usual to subject the hair to a special post-treatment. In this case, hair is conventionally treated with special active substances, for example quaternary ammonium salts, or specific polymers in the form of a rinse. Depending on formulation, this treatment improves the hair's combability, hold and body and reduces splitting.

In recent times, “combined preparations” have been developed in order to reduce the complexity of conventional multistage methods, in particular for direct use by consumers. In addition to the conventional components, for example for cleaning hair, these preparations also include active substances which were previously found only in hair post-treatment agents. The consumer is thus saved one stage during use; at the same time packaging costs are reduced because one less product is used.

Known active substances are, however, not capable of adequately meeting all requirements. A need accordingly still remains for active substances or active substance combinations for cosmetic agents which have good hair care properties and good biological degradability. In particular in surfactant- and/or electrolyte-containing formulations, there is a need for additional conditioning active substances which can be straightforwardly incorporated into known formulations and do not have their action attenuated therein due to incompatibility with other ingredients.

Silicones and, among these, amino-functional silicones are known as conditioning substances in hair treatment agents, and corresponding products are widespread on the market. There is, however, still a requirement to improve the effects achieved, in particular with regard to handle, combability, softness and volume of the hair or hairstyle and to reduce input quantities.

It is therefore desirable to provide silicone-containing hair treatment agents which impart better properties to hair treated therewith than do hair treatment agents with known amodimethicones. Furthermore, it is desirable to achieve equally good or better effects even with distinctly reduced input quantities. In particular, the products should improve the handle, combability, softness and volume of the hair or hairstyle and distinctly minimize the contact angle of water drops which come into contact with the treated hair, the contact angle being a measure of product performance.

Furthermore, other desirable features and characteristics of the present invention will become apparent from the subsequent detailed description of the invention and the appended claims, taken in conjunction with the accompanying drawings and this background of the invention.

### Brief Summary of the Invention

A cosmetic composition includes, in a cosmetically acceptable medium: (a) at least one thickener, selected from associative thickeners, crosslinked homopolymers of acrylic acid, crosslinked copolymers of (meth)acrylic acid and alkyl(C₁₋₆) acrylate, nonionic homopolymers and copolymers which include monomers with ethylenically unsaturated bonds of the ester and/or amide type, homopolymers of ammonium acrylate or copolymers of ammonium acrylate and acrylamide, fatty acid amides; and (b) at least one hydroxy-terminated organopolysiloxane of general formula (I),

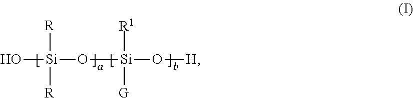

wherein R means a monovalent unsubstituted or halo-substituted hydrocarbon residue with 1 to 20 carbon atoms, R¹ means a monovalent unsubstituted or halo-substituted hydrocarbon residue with 1 to 20 carbon atoms, —OR⁴ or —OH, R⁴ means an alkyl residue with 1 to 6 carbon atoms, G means a group of general formula (II)

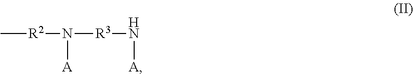

wherein R², R³ mutually independently mean a divalent hydrocarbon residue with 1 to 6 carbon atoms, wherein non-adjacent —CH₂ units may be replaced by units which are selected from —C(═O)—, —O—, and —S—, A means R⁵—C(═O)—, R⁵ means an alkyl residue with 1 to 20 carbon atoms, a means integral values of 100 to 1500 and b means integral values of at least 1.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention firstly provides a cosmetic composition including, in a cosmetically acceptable medium
(a) at least one thickener, selected from
   (i.) associative thickeners;
   (ii.) crosslinked homopolymers of acrylic acid;
   (iii.) crosslinked copolymers of (meth)acrylic acid and alkyl(C₁₋₆) acrylate;
   (iv.) nonionic homopolymers and copolymers which include monomers with ethylenically unsaturated bonds of the ester and/or amide type;
   (v.) homopolymers of ammonium acrylate or copolymers of ammonium acrylate and acrylamide;
   (vi.) fatty acid amides and
(b) at least one hydroxy-terminated organopolysiloxane of general formula (I),

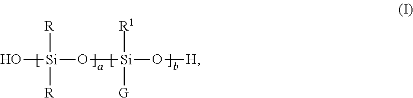

wherein
   R means a monovalent unsubstituted or halo-substituted hydrocarbon residue with 1 to 20 carbon atoms,
   R¹ means a monovalent unsubstituted or halo-substituted hydrocarbon residue with 1 to 20 carbon atoms, —OR⁴ or —OH,
   R⁴ means an alkyl residue with 1 to 6 carbon atoms,
   G means a group of general formula (II)

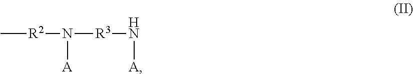

wherein
R², R³ mutually independently mean a divalent hydrocarbon residue with 1 to 6 carbon atoms, wherein non-adjacent —CH₂ units may be replaced by units which are selected from —C(═O)—, —O—, and —S—,
A means R⁵—C(═O)—,
R⁵ means an alkyl residue with 1 to 20 carbon atoms,
a means integral values of 100 to 1500 and
b means integral values of at least 1.

The agent according to the invention is a cosmetic agent. Depending on the area of application, it is thus possible to differentiate between a wide range of cosmetic agents, for example for skin conditioning (bath preparations, skin washing and cleaning agents, skin care agents, eye cosmetics, lip care agents, nail care agents, intimate hygiene agents, foot care agents), those with specific action (light protection agents, skin-tanning agents, depigmenting agents, deodorants, antiperspirants, depilatory agents, shaving agents, scents), those for dental and oral care (dental and oral care agents, denture care agents, prosthesis adhesives) and those for hair care (shampoos, hair care agents, hair setting agents, hair deforming agents, color-modifying agents).

Cosmetic agents which are preferred according to the invention are selected from the group of shower gels, shower foams, tooth cleaning agents, mouthwashes, shampoos, hair conditioners, conditioning shampoos, hair sprays, hair rinses, hair masks, hair packs, hair tonics, permanent wave fixing solutions, hair coloring shampoos, hair coloring agents, hair strengtheners, hair setting agents, hair styling preparations, blow drying lotions, mousse setting preparations, hair gels, hair waxes or combinations thereof.

More preferred cosmetic agents according to the invention serve to treat keratinic fibers and are thus hair treatment agents. Hair treatment agents for the purposes of the present invention are for example shampoos, hair conditioners, conditioning shampoos, hair sprays, hair rinses, hair masks, hair packs, hair tonics, permanent wave fixing solutions, hair coloring shampoos, hair coloring agents, hair strengtheners, hair setting agents, hair styling preparations, blow drying lotions, mousse setting preparations, hair gels, hair waxes or combinations thereof. In the light of the fact that men often shun using a number of different agents and/or a number of application steps, agents according to the invention are preferably those agents which a man uses in any event. Preferred agents according to the invention are therefore for example shampoos, conditioning agents or hair tonics.

The compositions of the invention have improved cosmetic properties (on hair for example lightness, softness, detanglability, natural feel and high-volume hairstyle, brightness) and the effects are moreover persistent and lasting. In particular, said effects are resistant to many shampoos.

Furthermore, when applied onto the skin (for example by a foam bath or shower gel), the compositions of the invention give rise to improved softness of the skin.

The agents according to the invention include as first essential ingredient at least one hydroxy-terminated organopolysiloxane of general formula (I),

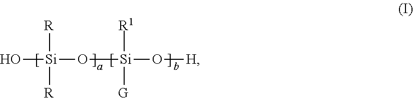

wherein
   R means a monovalent unsubstituted or halo-substituted hydrocarbon residue with 1 to 20 carbon atoms,
   R¹ means a residue R, —OR⁴ or —OH,
   R⁴ means an alkyl residue with 1 to 6 carbon atoms,
   G means a group of general formula (II)

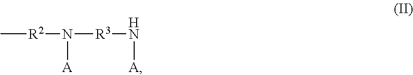

wherein
R², R³ mutually independently mean a divalent hydrocarbon residue with 1 to 6 carbon atoms, wherein non-adjacent —CH₂ units may be replaced by units which are selected from —C(═O)—, —O—, and —S—,
A means R⁵—C(═O)—,
R⁵ means an alkyl residue with 1 to 20 carbon atoms,
a means integral values of 100 to 1500 and
b means integral values of at least 1.

The individual siloxane units a and b in formula (I) and all the following formulae may be present as block copolymers or randomly distributed in each molecule; they are preferably randomly distributed.

As a result of the definitions for G and A, agents according to the invention include at least one hydroxy-terminated organopolysiloxane of general formula (Ia):

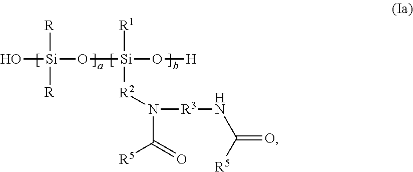

in which R, R¹, R², R³, R⁵, a and b are as defined above.

The residue R¹ may denote a monovalent unsubstituted or halo-substituted hydrocarbon residue with 1 to 20 carbon atoms, an OH group or a residue OR⁴, wherein R⁴ in turn denotes an alkyl residue with 1 to 6 carbon atoms. Preferred residues R¹ are methyl, ethyl-, n-propyl, isopropyl-, n-butyl-, isobutyl-, sec-butyl, tert-butyl, n-pentyl, n-hexyl-, hydroxy-, methoxy-, ethoxy-, n-propoxy and iso-propoxy residues. More preferred agents according to the invention include at least one hydroxy-terminated organopolysiloxane of general formula (Ia-1) and/or (Ia-2) and/or (Ia-3) and/or (Ia-4) and/or (Ia-5) and/or (Ia-6) and/or (Ia-7) and/or (Ia-8) and/or (Ia-9).

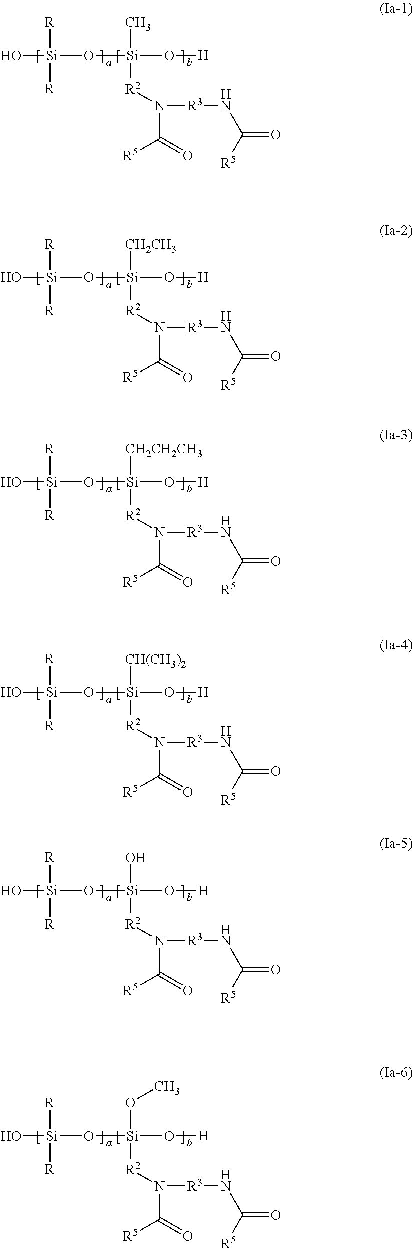

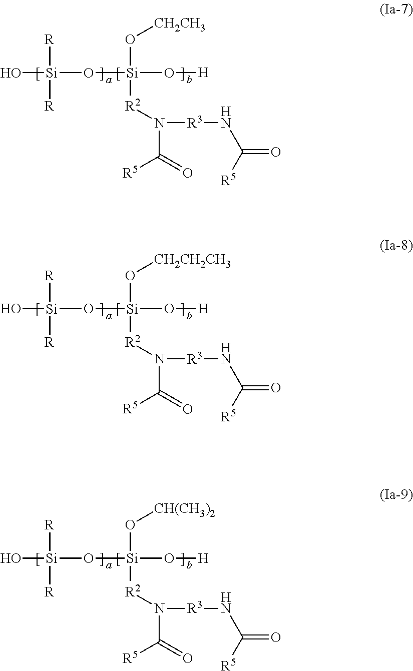

In more preferred cosmetic compositions according to the invention, R is selected from methyl, ethyl, n-propyl, isopropyl, n-butyl, isobutyl, sec-butyl, tert-butyl, n-pentyl, n-hexyl, phenyl residues, wherein R extremely preferably denotes methyl, ethyl or phenyl. Where R=methyl, formula (Ia) becomes formula (Ib), where R=ethyl it becomes formula (Ic), where R=phenyl it becomes formula (Id), such that more preferred agents according to the invention include at least one hydroxy-terminated organopolysiloxane of the general formula (Ib-1) and/or (Ib-2) and/or (Ib-3) and/or (Ib-4) and/or (Ib-5) and/or (Ib-6) and/or (Ib-7) and/or (Ib-8) and/or (Ib-9) and/or (Ic-1) and/or (Ic-2) and/or (Ic-3) and/or (Ic-4) and/or (Ic-5) and/or (Ic-6) and/or (Ic-7) and/or (Ic-8) and/or (Ic-9) and/or (Id-1) and/or (Id-2) and/or (Id-3) and/or (Id-4) and/or (Id-5) and/or (Id-6) and/or (Id-7) and/or (Id-8) and/or (Id-9).

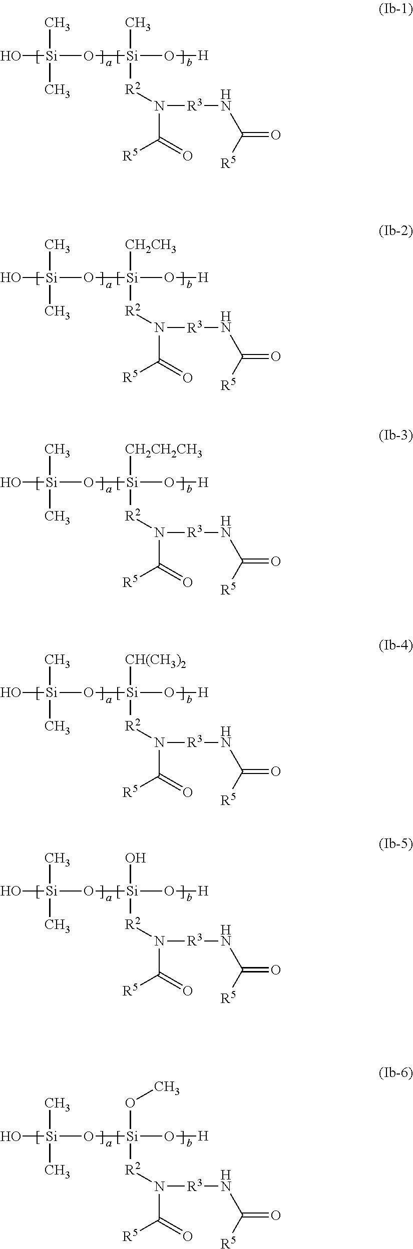

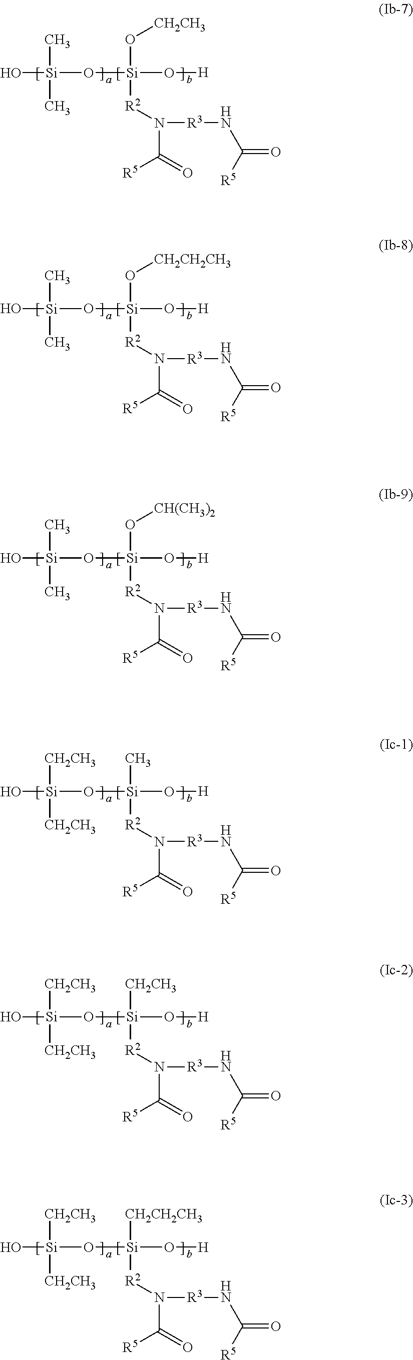

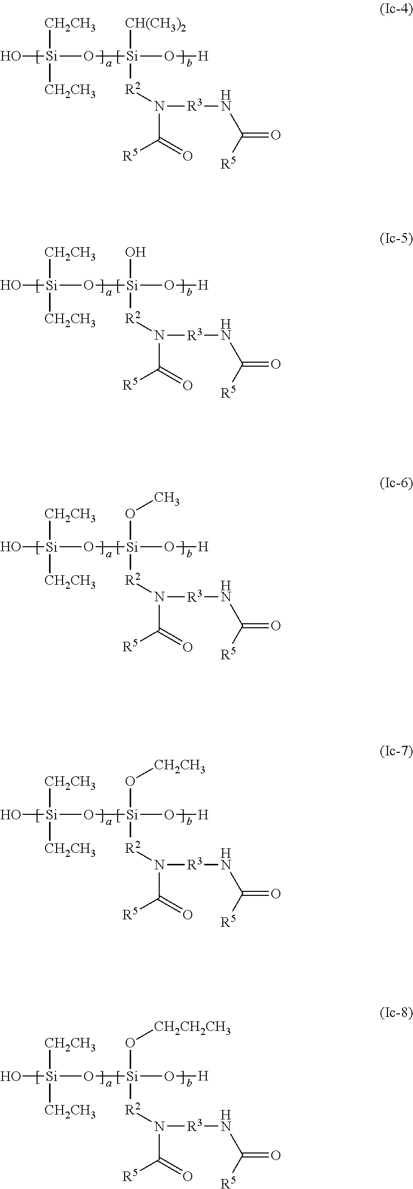

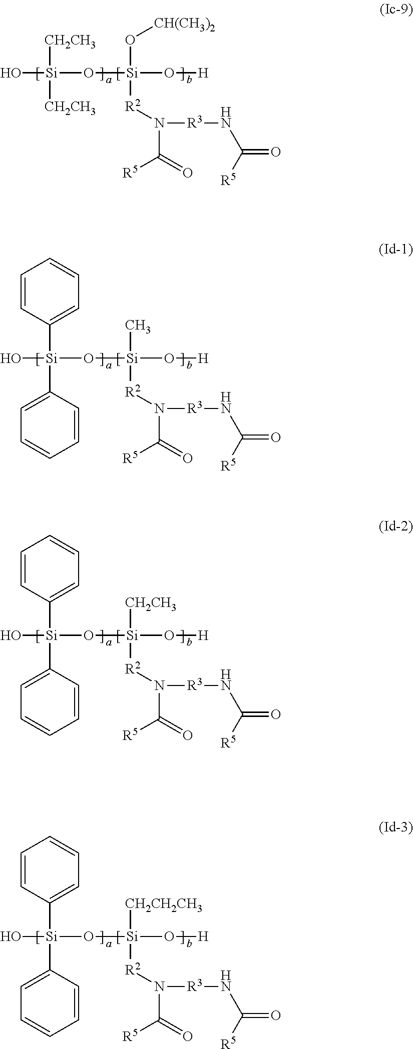

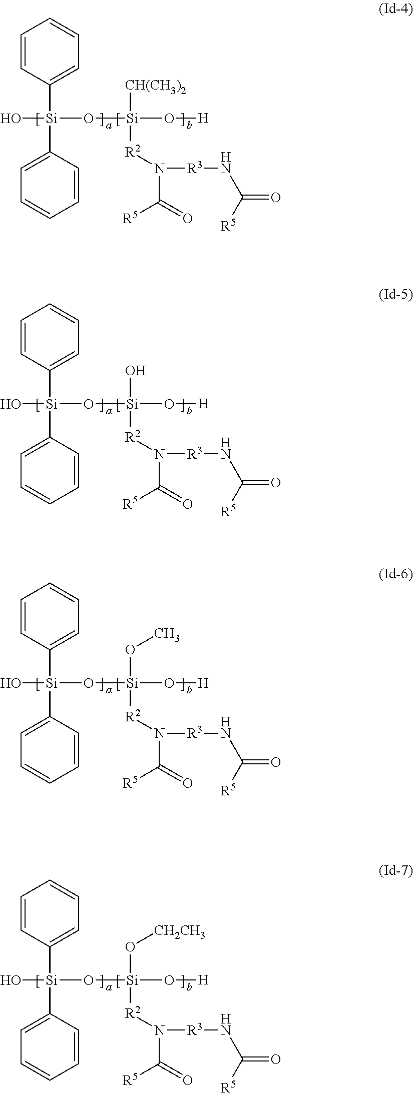

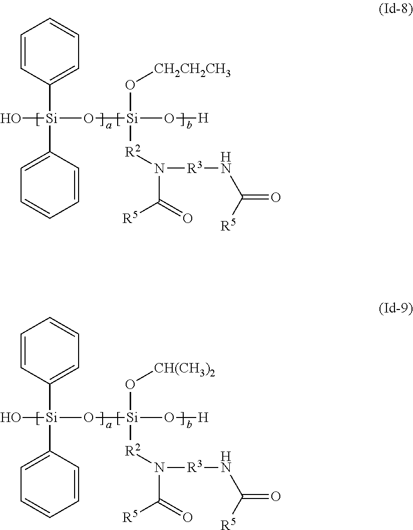

Preferred cosmetic compositions according to the invention include at least one hydroxy-terminated organopolysiloxane of general formula (I), in which the residues R², R³ are mutually independently selected from ethylene, n-propyl, iso-butylene or n-butylene residues.

R² particularly preferably denotes an n-propylene residue and R³ simultaneously denotes an ethylene residue, such that the grouping G is preferably a grouping

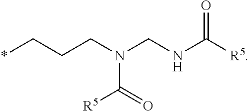

Particularly preferred agents according to the invention accordingly include at least one hydroxy-terminated organopolysiloxane of the general formula (Ie-1) and/or (Ie-2) and/or (Ie-3) and/or (Ie-4) and/or (Ie-5) and/or (Ie-6) and/or (Ie-7) and/or (Ie-8) and/or (Ie-9) with the definitions of R as stated for formula (I), wherein more preferred instances of R denote methyl, ethyl or phenyl residues:

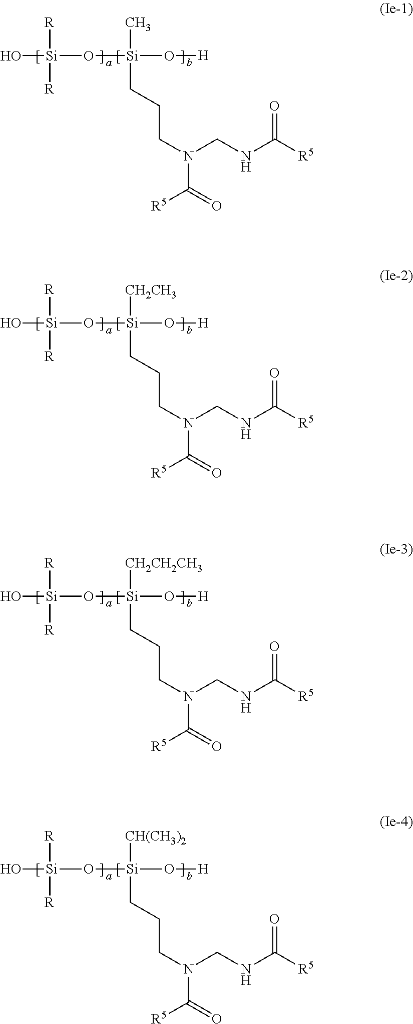

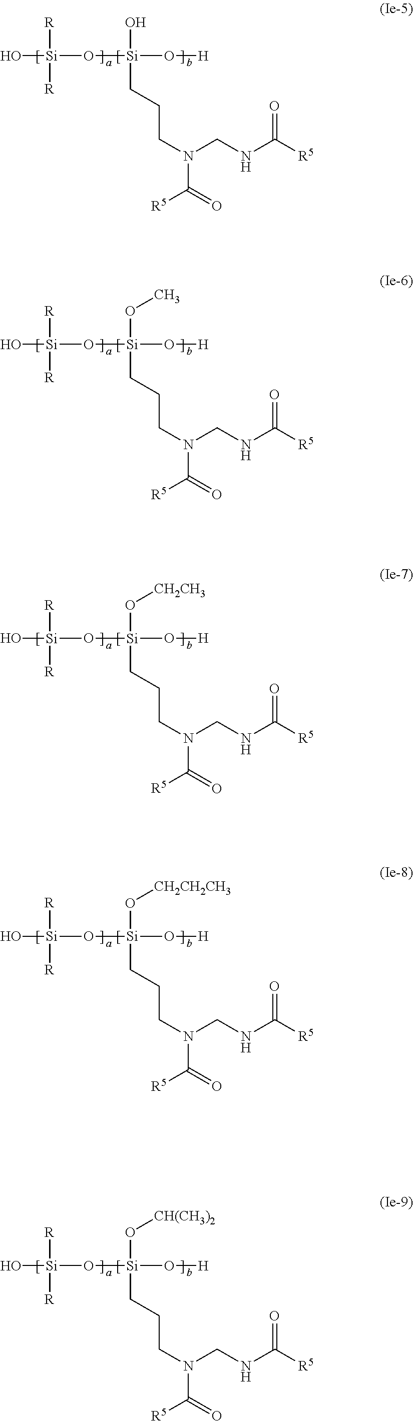

Particularly preferred cosmetic compositions according to the invention are characterized in that they include at least one hydroxy-terminated organopolysiloxane of the general formula (I) in which R⁵ is selected from methyl, ethyl, n-propyl, isopropyl, n-butyl, isobutyl, sec-butyl, tert-butyl, n-pentyl, n-hexyl residues, wherein R⁵ extremely preferably denotes methyl, ethyl, n-propyl or isopropyl.

Where R⁵=methyl, formula (Ie) becomes formula (If), where R⁵=ethyl it becomes formula (Ig), where R⁵=n-propyl it becomes formula (Ih), where R⁵=isopropyl it becomes formula (Ii), such that more preferred agents according to the invention include at least one hydroxy-terminated organopolysiloxane of general formula (If-4) and/or (If-5) and/or (If-6) and/or (If-7) and/or (If-8) and/or (If-9) and/or (Ig-1) and/or (Ig-2) and/or (Ig-3) and/or (Ig-4) and/or (Ig-5) and/or (Ig-6) and/or (Ig-7) and/or (Ig-8) and/or (Ig-9) and/or (Ih-1) and/or (Ih-2) and/or (Ih-3) and/or (Ih-4) and/or (Ih-5) and/or (Ih-6) and/or (Ih-7) and/or (Ih-8) and/or (Ih-9) and/or (Ii-1) and/or (Ii-2) and/or (Ii-3) and/or (Ii-4) and/or (Ii-5) and/or (Ii-6) and/or (Ii-7) and/or (Ii-8) and/or (Ii-9).

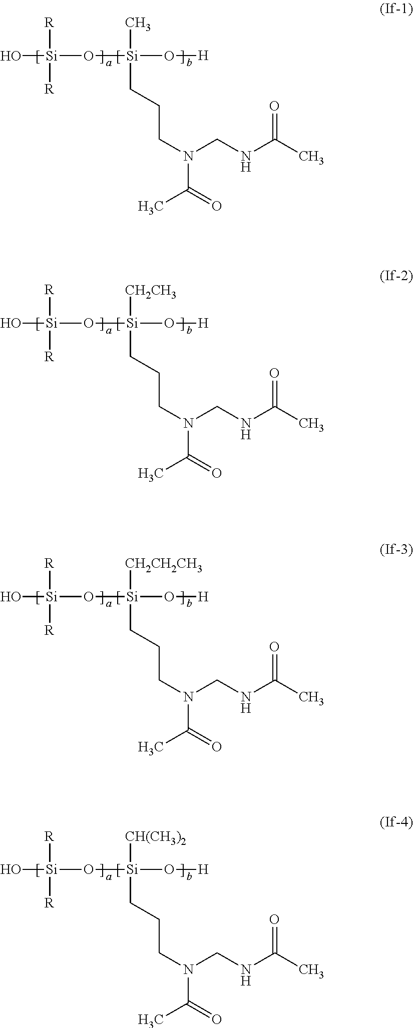

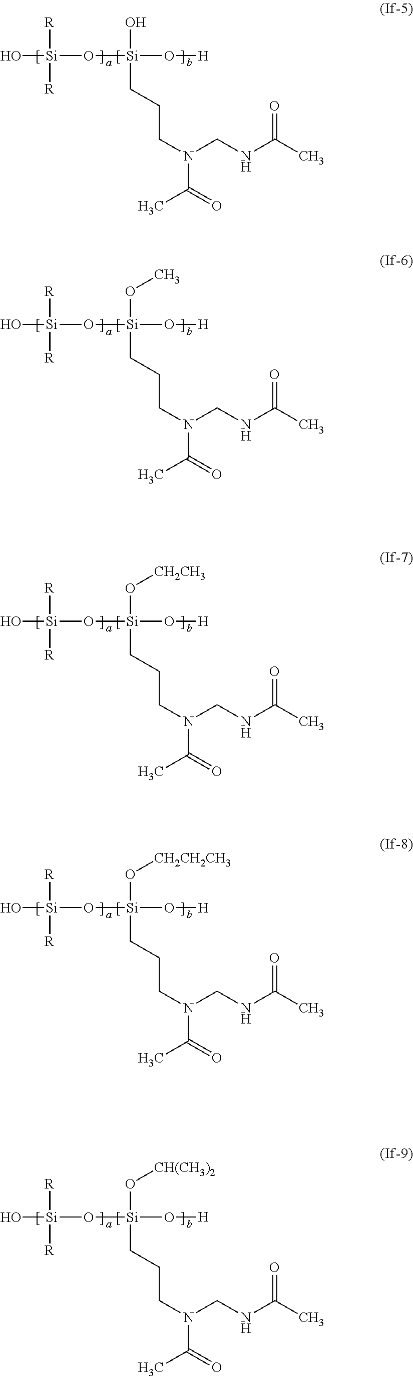

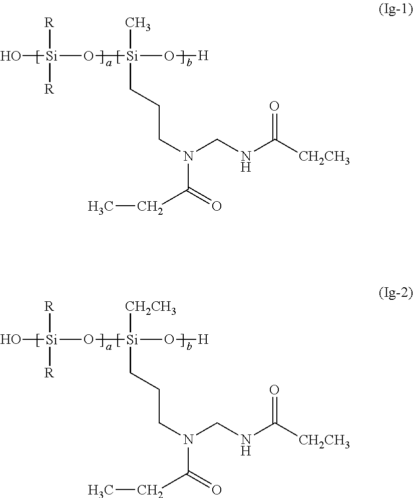

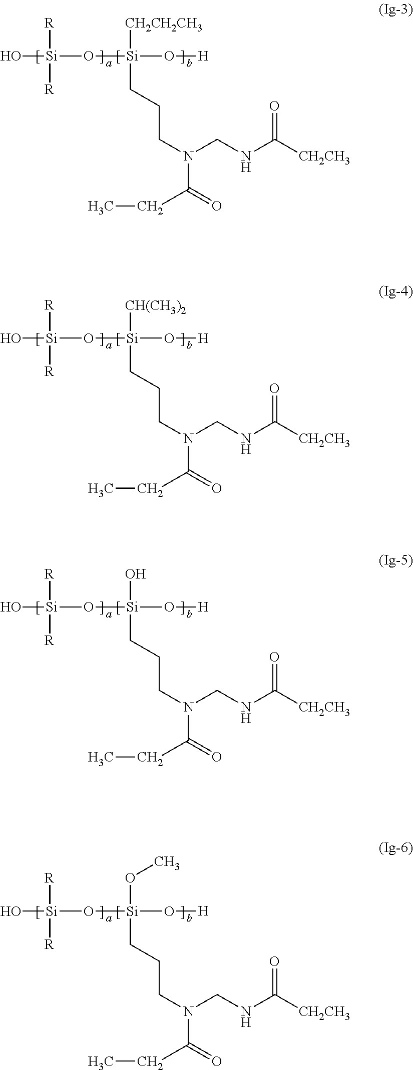

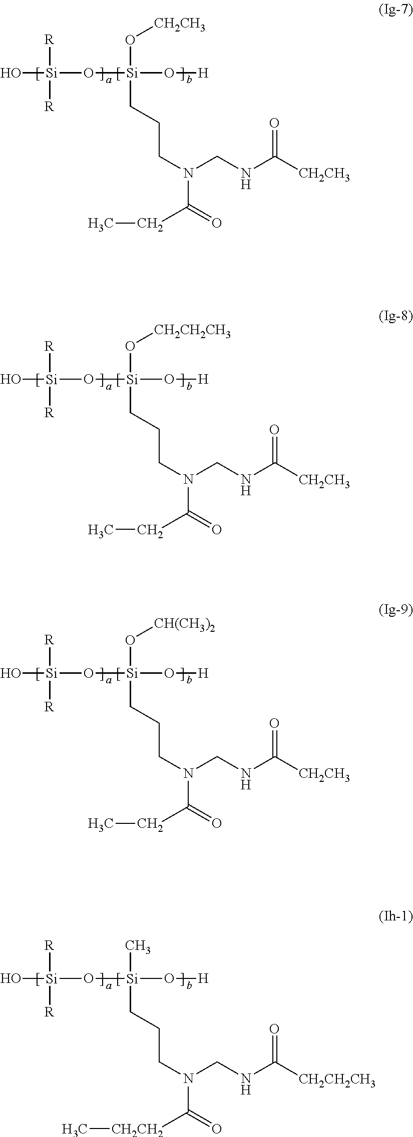

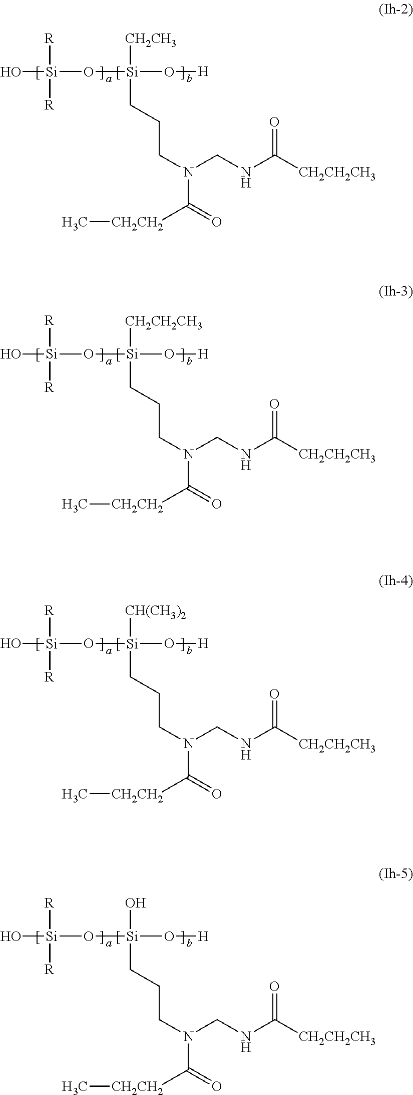

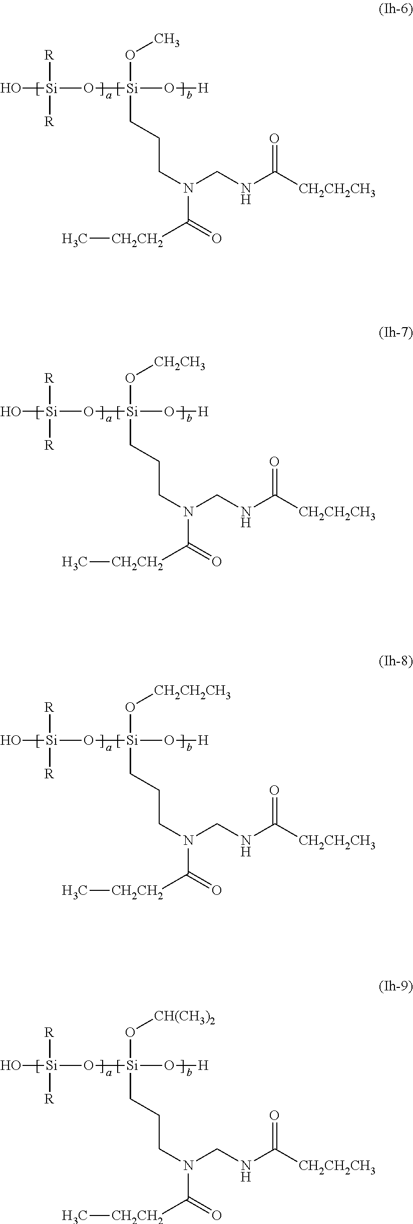

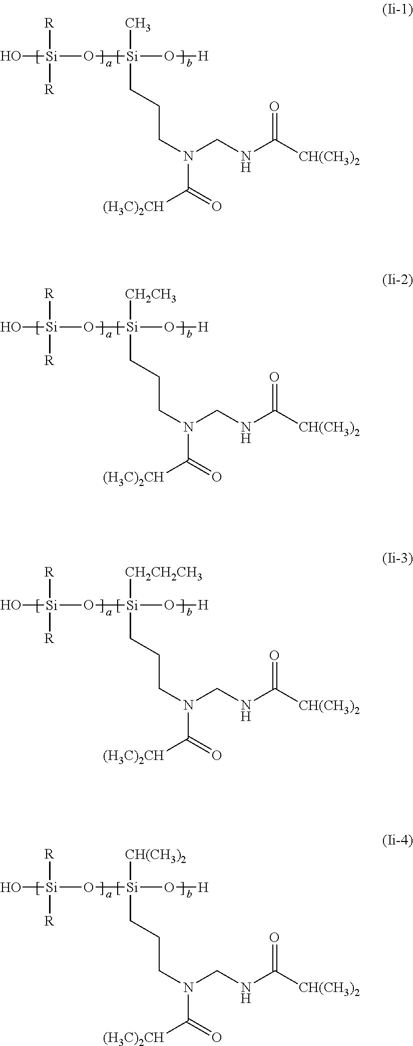

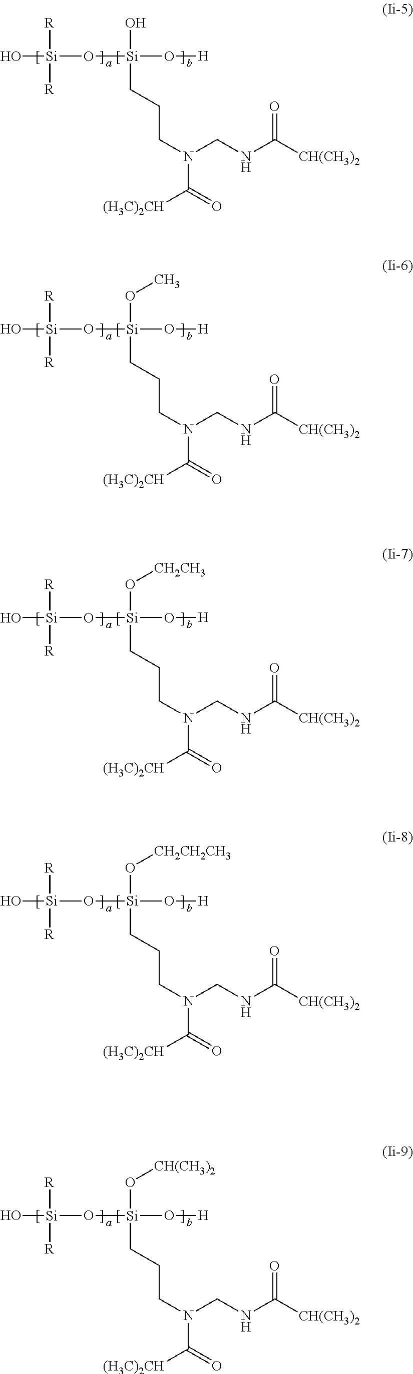

In all the above formulae, those representatives in which R denotes a methyl residue are more preferred.

In all the above formulae, the index denotes integral values of 100 to 1500. Preferably, a means integral values of at least 200, in particular at least 500 and at most 1300, more preferably at most 1100 and in particular at most 900. Agents according to the invention in which a assumes values of 220 to 910 are more preferred. In all the above formulae, the index b denotes integral values of at least 1. Preferably, b means integral values of at most 100, in particular at most 50, preferably at most 10 and in particular at most 5. Agents according to the invention in which b assumes the values 1, 2, 3, 4 or 5 are more preferred.

Preferably, a and b are selected such that the organopolysiloxane of formula (I) has, under standard conditions (20° C., 1013.25 mbar), a viscosity (Brookfield RTV, spindle 4, 20 rpm) of at least 100 mPa·s, preferably at least 1000 mPa·s, further preferably at least 5000 mPa·s and in particular at least 15,000 mPa·s. The viscosity preferably amounts to a maximum of 500,000 mPa·s, preferably at most 200,000 mPa·s, more preferably a maximum of 100,000 mPa·s and in particular at most 60,000 mPa·s. Agents according to the invention in which the organopolysiloxane of formula (I) has a viscosity of 17,000 to 55,000 mPa·s are more preferred.

The amine value of the organopolysiloxane of formula (I) preferably amounts to at least 0.001 mmol/g, more preferably at least 0.01 mmol/g. The amine value is more preferably at most 5 mmol/g, further preferably at most 1 mmol/g, still further preferably at most 0.1 mmol/g and in particular a maximum of 0.05 mmol/g. Agents according to the invention in which the organopolysiloxane of formula (I) has amine values of 0.015 to 0.045 mmol/g are more preferred.

Depending on the intended application of the agents according to the invention, the organopolysiloxane(s) of formula (I) may be used in varying quantities. Preferred cosmetic agents according to the invention are characterized in that, relative to the weight thereof, they include 0.00001 to 10 wt. %, preferably 0.0001 to 7.5 wt. %, further preferably 0.001 to 5 wt. %, further preferably 0.01 to 3 wt. % and in particular 0.1 to 1 wt. % of organopolysiloxane(s) of formula (I).

It has been found that the action of the silicones used according to the invention may be still further increased if specific nonionic components are likewise used in the agents according to the invention. These nonionic components furthermore have positive effects on the storage stability of the agents according to the invention. Nonionic components which are particularly suitable here are ethoxylates of decanol, undecanol, dodecanol, tridecanol etc. Ethoxylated tridecanols have proven particularly suitable and are more preferentially incorporated into the agents according to the invention. Cosmetic compositions which are more preferred according to the invention include, relative to the weight thereof, 0.00001 to 5 wt. %, preferably 0.0001 to 3.5 wt. %, more preferably 0.001 to 2 wt. %, further preferably 0.01 to 1 wt. % and in particular 0.1 to 0.5 wt. % of branched, ethoxylated tridecanol (INCI name: Trideceth-5) or α-iso-tridecyl-ω-hydroxy polyglycol ether (INCI name: Trideceth-10) or mixtures thereof.

Organopolysiloxane(s) of formula (I) which are preferred according to the invention have both hydroxyl and alkoxy groups. Cosmetic compositions which are more preferred according to the invention include organopolysiloxane(s) of formula (I) in which the hydroxy/alkoxy molar ratio is in the range from 0.2:1 to 0.4:1, preferably in the range from 1:0.8 to 1:1.1. The average molecular weights of the organopolysiloxane(s) of formula (I) preferably amount to from 2,000 to 200,000 and still more preferably from 5,000 to 100,000, in particular 10,000 to 50,000 dalton. Cosmetic compositions in which the weight-average molar mass of the organopolysiloxane(s) of formula (I) present therein is in the range from 2,000 to 1,000,000 gmol⁻¹, preferably in the range from 5,000 to 200,000 gmol⁻¹, are preferred.

The average molecular weights of amino-substituted silicones may for example be measured by gel permeation chromatography (GPC) at room temperature in polystyrene. Columns which may be selected are μ-Styragel columns, with THF as eluent and a flow rate of 1 ml/min. Detection preferably proceeds by refractometry and UV meter. The organopolysiloxane(s) of formula (I) are preferably used as an oil-in-water emulsion. The oil-in-water emulsion may include one or more surfactants. The surfactants may be of any desired kind, preferably cationic and/or nonionic. The number-average size of the silicone droplets in the emulsion is preferably between 3 nm and 500 nm, more preferably between 5 nm and 60 nm (inclusive) and in particular between 10 nm and 50 nm (inclusive).

Cosmetic compositions according to the invention in which the organopolysiloxane(s) of formula (I) are present in the form of an oil-in-water emulsion, in which the number-average size of the silicone particles in the emulsion is in the range from 3 to 500 nm, preferably in the range from 5 to 60 nm, are preferred according to the invention.

A further essential component of the compositions according to the invention is a thickener, the function of which is to increase the viscosity of the composition. These thickeners are selected from
(i.) associative thickeners;
(ii.) crosslinked homopolymers of acrylic acid;
(iii.) crosslinked copolymers of (meth)acrylic acid and alkyl(C₁₋₆) acrylate;
(iv.) nonionic homopolymers and copolymers which include monomers with ethylenically unsaturated bonds of the ester and/or amide type;
(v.) homopolymers of ammonium acrylate or copolymers of ammonium acrylate and acrylamide;
(vi.) fatty acid amides.

“Associative thickeners” according to the invention are thickeners with both hydrophilic and hydrophobic units, in particular with at least one C₈-C₃₀ fatty chain and at least one hydrophilic unit.

Cosmetic compositions which are preferred according to the invention include as associative thickener an associative polymer which is selected from
(i.) nonionic amphiphilic polymers which include at least one fatty chain and at least one hydrophilic unit;
(ii.) anionic amphiphilic polymers which include at least one hydrophilic unit and at least one unit with a fatty chain;
(iii.) cationic amphiphilic polymers which include at least one hydrophilic unit and at least one unit with a fatty chain;
(iv.) amphoteric amphiphilic polymers which include at least one hydrophilic unit and at least one unit with a fatty chain,
wherein the fatty chains have 10 to 30 carbon atoms.

The nonionic amphiphilic polymer with at least one fatty acid chain and at least one hydrophilic unit is preferably selected from:
(1) celluloses which are modified with groups which have at least one fatty chain;
(2) hydroxypropyl guar compounds which are modified with groups which have at least one fatty chain;
(3) urethane polyethers which include at least one fatty chain, such as alkyl or alkenyl groups with 10 to 30 carbon atoms;
(4) copolymers of vinylpyrrolidone and hydrophobic monomers with a fatty chain;
(5) copolymers of alkyl(C₁₋₆) methacrylates or alkyl(C₁₋₆) acrylates and amphiphilic monomers which include at least one fatty chain;
(6) copolymers of hydrophilic methacrylates or acrylates and hydrophobic monomers which include at least one fatty chain.

Examples of (1) are hydroxyethylcelluloses with groups which include at least one fatty chain, such as alkyl, arylalkyl, alkylaryl, or mixtures thereof, wherein the alkyl groups are preferably selected from C₈-C₂₂, such as the product Natrosol Plus Grade 330 CS (C₁₆ alkyl) from Aqualon, or the product Bermocoll EHM 100 from Berol Nobel, or alkylphenols provided with polyalkylene glycol ether groups, such as the product AMERCELL POLYMER HM-1500 (polyethylene glycol (15) nonylphenol ether sulfate) from Amerchol.

Examples of (2) are the product Esaflor HM 22 (C₂₂ alkyl chain) from Lamberti, MiraCare XC95-3 (C₁₄ alkyl chain) and RE205-1 (C₂₀ alkyl chain) from Rhodia Chimie.

Examples of (3) are the products Dapral T 210 and T 212 Dapral from Akzo or the products Aculyn 44 and 46 from Rohm & Haas.

Examples of (4) are Antaron V216 or V216 GANEX (vinyl-pyrrolidone/hexadecene copolymer) from ISP and Antaron V220 or V220 GANEX (vinylpyrrolidone/eicosene) from ISP.

One example of (5) is the oxyethylenated copolymer of methyl methacrylate/stearyl acrylate which is distributed by Goldschmidt under the name Antil 208.

One example of (6) is the copolymer of polyethylene glycol methacrylate/lauryl methacrylate.

Cosmetic compositions which are preferred according to the invention are therefore characterized in that the nonionic amphiphilic polymers which include at least one fatty chain and at least one hydrophilic unit are selected from
(1) celluloses which are modified with groups which have at least one fatty chain;
(2) hydroxypropyl guar compounds which are modified with groups which have at least one fatty chain;
(3) urethane polyethers which include at least one fatty chain, such as alkyl or alkenyl groups with 10 to 30 carbon atoms;
(4) copolymers of vinylpyrrolidone and hydrophobic monomers with a fatty chain;
(5) copolymers of alkyl(C₁₋₆) methacrylates or alkyl(C₁₋₆) acrylates and amphiphilic monomers which include at least one fatty chain;
(6) copolymers of hydrophilic methacrylates or acrylates and hydrophobic monomers which include at least one fatty chain.

Among the anionic amphiphilic polymers of the invention with at least one hydrophilic and at least one fatty chain, those which are preferred are those with at least one allyl ether unit with a fatty chain and at least one hydrophilic unit prepared from an ethylenically unsaturated anionic monomer, in particular acrylic acid, methacrylic acid or mixtures thereof.

More preferred are allyl ethers with a fatty chain corresponding to the monomer of formula (M-III):

CH₂═C(R¹)CH₂—OBₙR (M-III)

wherein R¹ is —H or —CH₃, B denotes an ethylene oxide grouping, n means zero or an integer between 1 and 100, R represents a hydrocarbon residue selected from alkyl, arylalkyl, aryl, alkylaryl, cycloalkyl with 10 to 30 C atoms, preferably 10 to 24 and even more particularly 12 to 18 carbon atoms.

More preferred units of formula (M-III) are those in which R¹═—H, n=10, and R is a stearyl residue (C₁₈).

Among these anionic amphiphilic polymers those which are more preferred are acrylates with 20 to 60 wt. % of acrylic acid and/or methacrylic acid, 5 to 60 wt. % of lower alkyl (meth)acrylates, from 2 to 50 wt. % of allyl ethers with a fatty chain of formula (M-III) and 0 to 1 wt. % of crosslinking agent. A copolymer of diallyl phthalate, (meth)acrylate, allyl-divinylbenzene dimethacrylate, (poly)ethylene glycol and methylenebisacrylamide is for example conventionally commercial.

More preferred terpolymers of methacrylic acid, ethyl acrylate, polyethylene glycol (10 EO) stearyl alcohol ether (Steareth-10) are sold under the names SALCARE SC 80 and SALCARE SC90 which are 30% aqueous emulsions of a crosslinked terpolymer of methacrylic acid, ethyl acrylate and Steareth-10 allyl ether (40/50/10).

Preferred cosmetic compositions according to the invention are characterized in that the anionic amphiphilic polymers which include at least one hydrophilic unit and at least one unit with a fatty chain are selected from compounds which include at least one allyl ether unit with a fatty chain and at least one hydrophilic unit, compounds synthesized from an ethylenically unsaturated, anionic monomer, compounds which include at least one hydrophilic unit of the type of an olefinically unsaturated carboxylic acid and at least one hydrophobic unit exclusively of the type of an alkyl(C₁₋₃₀) ester of an unsaturated carboxylic acid, and methacrylic acid/methyl acrylate/dimethyl-meta-isopropenylbenzyl isocyanate copolymers of an ethoxylated alcohol.

The anionic amphiphilic polymers may further be selected from those which include at least one hydrophilic unit of olefinically unsaturated carboxylic acids and at least one hydrophobic unit of the (C₁₀-C₃₀) alkyl ester of an unsaturated carboxylic acid type. The hydrophilic unit of the olefinically unsaturated carboxylic acid type corresponds for example to the monomer of formula (M-IV):

H₂C═C(R¹)C(O)—OH (M-IV)

in which R¹ is selected from —H, —CH₃ and —C₂H₅, i.e. acrylic acid, methacrylic acid and ethacrylic acid units. While the hydrophobic unit of the (C₁₀-C₃₀) alkyl ester of an unsaturated carboxylic acid type corresponds for example to the monomer of formula (V):

H₂C═C(R¹)C(O)—OR² (M-V)

in which R¹ is selected from —H, —CH₃ and —C₂H₅ (i.e. acrylate, methacrylate and ethacrylate units), R² is selected from C₁₀-C₃₀ alkyl residues, for example C₁₂-C₂₂ alkyl residue.

Examples of (C₁₀-C₃₀) alkyl esters of unsaturated carboxylic acids are lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Representative anionic amphiphilic polymers which may be used are also polymers prepared from a mixture of monomers comprising

(i) acrylic acid esters of formula (M-VI)

H₂C═C(R¹)C(O)—OR² (M-VI)

in which R¹ is selected from —H, —CH₃ (i.e. acrylate, methacrylate units), R² is selected from C₁₀-C₃₀ alkyl residues, for example C₁₂-C₂₂ alkyl residue and a crosslinking agent; such as polymers of 95% to 60 wt. % of acrylic acid (hydrophilic unit), 4% to 40 wt. % of C₁₀-C₃₀ alkyl acrylate (hydrophobic unit) and 0% to 6 wt. % of crosslinking-polymerizable monomers or polymers derived from 98% to 96% of the weight of the acrylic acid (hydrophilic unit), 1% to 4 wt. % of C₁₀-C₃₀ alkyl acrylate (hydrophobic unit) and 0.1% to 0.6 wt. % of crosslinking-polymerizable monomers or
(ii) acrylic acid and lauryl methacrylate, such as the polymers which are formed from 66 wt. % of acrylic acid and 34 wt. % of lauryl methacrylate.

The crosslinking agent may be a monomer which includes a group with at least one other polymerizable group, the unsaturated bonds of which are not conjugated to one another. Polyallyl ethers such as polyallylsucrose and polyallylpentaerythritol may be mentioned by way of example. Among the above-mentioned polymers, those which are preferred are for example the products from Goodrich under the trade names Pemulen TR1, Pemulen TR2, Carbopol 1382 and others, for example, Pemulen TR1 and the product Coatex SX from Seppic.

Among anionic amphiphilic fatty acid chain polymers which may also be mentioned are the methacrylic acid/methyl acrylate/ethoxylated alkyl dimethyl-meta-isopropenylbenzylisocyanate copolymers which are distributed by Amerchol under the name Viscophobe DB 1000.

Cationic amphiphilic polymers may also be used, for example prepared from quaternized cellulose derivatives and polyacrylates with amino side groups.

The quaternized cellulose derivatives are for example formed from selected quaternized celluloses with groups which include at least one fatty chain, such as alkyl, arylalkyl and alkylaryl groups with at least 8 C atoms and mixtures thereof. Quaternized hydroxyethylcellulose with groups which include at least one fatty chain, such as alkyl, arylalkyl and alkylaryl groups with at least 8 C atoms and mixtures thereof are preferred. Quaternized and non-quaternized polyacrylates with amino side groups and for example hydrophobic groups, such as Steareth-20 (polyoxyethylenated (20) stearyl alcohol) and (C₁₀-C₃₀)-alkyl PEG-20 itaconate are likewise preferred.

The alkyl residues of the above-stated quaternized celluloses and hydroxyethylcelluloses include for example 8 to 30 carbon atoms. The aryl residues are for example selected from phenyl, benzyl, naphthyl and anthryl groups. Examples of quaternized alkylhydroxyethylcelluloses prepared from C₈-C₃₀ fatty acid chains are the products Quatrisoft LM 200, LM-X Quatrisoft 529-18-A, Quatrisoft LM-X 529-18B (C₁₂ alkyl) and Quatrisoft LM-X 529-8 (C₁₈) sold by Amerchol, and the products Crodacel QM, Crodacel QL (Cu alkyl) and Crodacel QS (C₁₈) from Croda. Examples of polyacrylates with amino side chains are the polymers 8781-124B or 9492-103 and Structure Plus from National Starch.

Among amphoteric amphiphilic polymers with at least one hydrophilic unit and at least one fatty acid chain unit which may be mentioned are for example methacrylamidopropyltrimethylammonium/acrylic acid/C₁₀-C₃₀ alkyl methacrylate copolymers, wherein the alkyl residue is for example a stearyl residue.

The associative thickeners in the cosmetic compositions may for example be used in solution or in dispersion with a concentration of 1% active material in water and with a viscosity, measured with a Rheomat RM 180 rheometer at 25° C., which is greater than 0.1 cps and preferably greater than 0.2 cp, at a shear rate of 200 s⁻¹.

To summarize, preferred cosmetic compositions according to the invention are characterized in that the cationic amphiphilic polymers are selected from quaternized cellulose derivatives and polyacrylates with aminated side chains.

Further preferred cosmetic compositions are characterized in that the amphoteric amphiphilic polymers which include at least one fatty chain are selected from the copolymers of methacrylamidopropyltrimethylammonium chloride/acrylic acid/alkyl(C₁₀₋₃₀) methacrylate.

(ii) Among the crosslinked acrylic acid homopolymers which may be mentioned are those with an allyl alcohol ether of the sugar series, such as the products sold under the name Carbopol 980, 981, 954, 2984 and 5984 by Goodrich, or the products sold under the names Synthalen M and Synthalen K by 3 VSA.
(iii) Crosslinked copolymers of (meth) acrylic acid and C₁-C₆ alkyl acrylates may be selected from crosslinked copolymers of methacrylic acid and ethyl acrylate in the form of an aqueous dispersion which includes 38% active material. These are sold under the names Viscoatex 538C by Coatex. Crosslinked copolymers of acrylic acid and ethyl acrylate in the form of an aqueous dispersion which includes 28% active material are distributed under the name Aculyn 33 by Rohm & Haas. Crosslinked copolymers of methacrylic acid and ethyl acrylate comprise an aqueous dispersion with 30% active material produced and sold under the name Carbopol Aqua SF-1 by Noveon.
(iv) Nonionic homo- or copolymers prepared from ethylenically unsaturated monomers of the ester and/or amide type which may be mentioned are: Cyanamer P250 from Cytec (polyacrylamide); PMMA MBX-8C from US Cosmetics (methyl methacrylate/ethylene glycol dimethacrylate copolymer); Acryloid B66 from Rohm & Haas (butyl methacrylate/methyl methacrylate copolymer), BPA 500 from Kobo (polymethyl methacrylate).
(v) Ammonium acrylate homopolymers which may be mentioned are the product Microsap PAS 5193 from Hoechst.

Copolymers of ammonium acrylate and of acrylamide are distributed as a product under the name Bozepol C Nouveau or product PAS 5193 by Hoechst.

Cosmetic compositions which are preferred according to the invention include the thickener(s) in a total quantity of 0.001 to 20 wt. %, preferably of 0.01 to 15 wt. %, further preferably of 0.1 to 10 wt. % and in particular of 0.25 to 5 wt. %, relative to the total weight of the composition.

Depending on the intended application, the agents according to the invention include further essential ingredients. Cleaning or conditioning compositions such as for example shampoos or conditioners include at least one surfactant, wherein, depending on the area of application, surface-active substances are known as surfactants or as emulsifiers and are selected from anionic, cationic, zwitterionic, ampholytic and nonionic surfactants and emulsifiers.

Cosmetic agents which are preferred according to the invention are characterized in that, relative to the weight thereof, they include 0.5 to 70 wt. %, preferably 1 to 60 wt. % and in particular 5 to 25 wt. % of anionic and/or nonionic and/or cationic and/or amphoteric surfactant(s).

Anionic surfactants and emulsifiers which are suitable in the compositions according to the invention are any anionic surface-active substances suitable for use on the human body. These are characterized by an anionic water-solubilizing group such as for example a carboxylate, sulfate, sulfonate or phosphate group and a lipophilic alkyl group with approximately 8 to 30 C atoms. The molecule may additionally include glycol or polyglycol ether groups, ester, ether and amide groups and hydroxyl groups. Examples of suitable anionic surfactants and emulsifiers are, in each case in the form of sodium, potassium and ammonium and the mono-, di- and trialkanolammonium salts with 2 to 4 C atoms in the alkanol group,
linear and branched fatty acids with 8 to 30 C atoms (soaps),
ether carboxylic acids of formula R—O—(CH₂—CH₂O)ₓ—CH₂—COOH, in which R is a linear alkyl group with 8 to 30 C atoms and x=0 or 1 to 16,
acyl sarcosides with 8 to 24 C atoms in the acyl group,
acyl taurides with 8 to 24 C atoms in the acyl group,
acyl isethionates with 8 to 24 C atoms in the acyl group,
linear alkane sulfonates with 8 to 24 C atoms,
linear alpha-olefin sulfonates with 8 to 24 C atoms,
alpha-sulfofatty acid methyl esters of fatty acids with 8 to 30 C atoms,
acyl glutamates of formula (I),

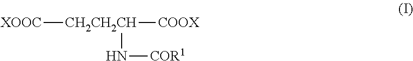

in which R¹CO denotes a linear or branched acyl residue with 6 to 22 carbon atoms and 0, 1, 2 or 3 double bonds and X denotes hydrogen, an alkali metal and/or alkaline earth metal, ammonium, alkylammonium, alkanolammonium or glucammonium, for example acyl glutamates which are derived from fatty acids with 6 to 22, preferably 12 to 18 carbon atoms, such as for example C_{12/14} or C_{12/18} coconut fatty acid, lauric acid, myristic acid, palmitic acid and/or stearic acid, in particular sodium N-cocoyl L-glutamate and sodium N-stearoyl L-glutamate, esters of a hydroxy-substituted di- or tricarboxylic acid of general formula (II),

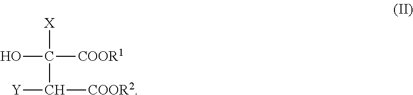

in which X═H or a —CH₂COOR group, Y═H or —OH under the condition that Y ═H, if X═—CH₂COOR, R, R¹ and R² mutually independently mean a hydrogen atom, an alkali or alkaline earth metal cation, an ammonium group, the cation of an organoammonium base or a residue Z which originates from a polyhydroxylated organic compound which is selected from the group of etherified (C₆-C₁₈) alkyl polysaccharides with 1 to 6 monomeric saccharide units and/or of etherified aliphatic (C₆-C₁₆) hydroxyalkyl polyols with 2 to 16 hydroxyl residues, with the proviso that at least one of the groups R, R¹ or R² is a residue Z,
esters of sulfosuccinic acid or sulfosuccinates of general formula (III),

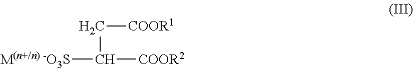

in which M⁽ⁿ⁺ⁿ⁾ represents, where n=1, a hydrogen atom, an alkali metal cation, an ammonium group or the cation of an organoammonium base and, where n=2, an alkaline earth metal cation and R¹ and R² mutually independently mean a hydrogen atom, an alkali or alkaline earth metal cation, an ammonium group, the cation of an organoammonium base or a residue Z which originates from a polyhydroxylated organic compound which is selected from the group of etherified (C₆-C₁₈) alkyl polysaccharides with 1 to 6 monomeric saccharide units and/or of etherified aliphatic (C₆-C₁₆) hydroxyalkyl polyols with 2 to 16 hydroxyl residues, with the proviso that at least one of the groups R¹ or R² is a residue Z,
sulfosuccinic acid mono- and dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
alkyl sulfates and alkyl polyglycol ether sulfates of formula R—O(CH₂—CH₂O)ₓ—OSO₃H, in which R is a preferably linear alkyl group with 8 to 30 C atoms and x=0 or 1 to 12,
mixed surface-active hydroxysulfonates according to DE-A-37 25 030, which is hereby incorporated by reference,
esters of tartaric acid and citric acid with alcohols which are addition products of approximately 2-15 molecules of ethylene oxide and/or propylene oxide onto C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants and emulsifiers are acyl glutamates, acyl isethionates, acyl sarcosinates and acyl taurates, in each case with a linear or branched acyl residue with 6 to 22 carbon atoms and 0, 1, 2 or 3 double bonds, which residue is selected in more preferred embodiments from an octanoyl, decanoyl, lauroyl, myristoyl, palmitoyl and stearoyl residue, esters of tartaric acid, citric acid or succinic acid or of the salts of these acids with alkylated glucose, in particular the products with the INCI names Disodium Coco-Glucoside Citrate, Sodium Coco-Glucoside Tartrate and Disodium Coco-Glucoside Sulfosuccinate, alkyl polyglycol ether sulfates and ether carboxylic acids with 8 to 18 C atoms in the alkyl group and up to 12 ethoxy groups per molecule, sulfosuccinic acid mono- and dialkyl esters with 8 to 18 C atoms in the alkyl group and sulfosuccinic acid monoalkyl polyoxyethyl esters with 8 to 18 C atoms in the alkyl group and 1 to 6 ethoxy groups.

Those surface-active compounds which bear at least one quaternary ammonium group and at least one —COO⁽⁻⁾ or —SO₃⁽⁻⁾ group on each molecule are designated as zwitterionic surfactants and emulsifiers. Particularly suitable zwitterionic surfactants and emulsifiers are “betaines” such as N-alkyl N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines with in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Preferred anionic surfactants are alkyl sulfates, alkyl polyglycol ether sulfates and ether carboxylic acids with 10 to 18 C atoms in the alkyl group and up to 12 glycol ether groups per molecule, sulfosuccinic acid mono- and dialkyl esters with 8 to 18 C atoms in the alkyl group and sulfosuccinic acid monoalkyl polyoxyethyl esters with 8 to 18 C atoms in the alkyl group and 1 to 6 oxyethyl groups.

More preferred anionic surfactants are the alkali metal or ammonium salts of lauryl ether sulfate with a degree of ethoxylation of 2 to 4 EO.

More preferred cosmetic agents according to the invention are characterized in that, relative to the weight thereof, they include 0.1 to 20 wt. %, preferably 0.25 to 17.5 wt. % and in particular 5 to 15 wt. % of anionic surfactant(s), more preferably fatty alcohol ether sulfates of formula

H₃C—(CH₂)ₙ—(OCH₂CH₂)ₖ—OSO₃⁻M⁺

in which n denotes values of 5 to 21, preferably of 7 to 19, more preferably of 9 to 17 and in particular of 11 to 13 and k denotes values of 1, 2, 3, 4, 5, 6, 7, 8, 9 or 10, preferably 1, 2 or 3 and in particular 2, and M denotes a cation from the group Na⁺, K⁺, NH⁴⁺, ½Mg²⁺, ½Zn²⁺, preferably Na⁺.

Those surface-active compounds which bear at least one quaternary ammonium group and at least one —COO⁽⁻⁾ or —SO₃⁽⁻⁾ group on each molecule are designated as zwitterionic surfactants and emulsifiers. Particularly suitable zwitterionic surfactants and emulsifiers are “betaines” such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines with in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Ampholytic surfactants and emulsifiers are taken to mean those surface-active compounds which, in addition to a C₈-C₂₄ alkyl or acyl group, include at least one free amino group and at least one —COOH or —SO₃H group and are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids with in each case approximately 8 to 24 C atoms in the alkyl group. More preferred ampholytic surfactants are N-cocoalkyl aminopropionate, cocoacylaminoethyl aminopropionate and C₁₂₋₁₈ acyl sarcosine.

More preferred cosmetic agents according to the invention are characterized in that they include amphoteric surfactant(s) from the groups of
N-alkylglycines,
N-alkylpropionic acids,
N-alkylaminobutyric acids,
N-alkyliminodipropionic acids,
N-hydroxyethyl-N-alkylamidopropylglycines,
N-alkyltaurines,
N-alkylsarcosines,
2-alkylaminopropionic acids with in each case approximately 8 to 24 C atoms in the alkyl group,
alkylaminoacetic acids with in each case approximately 8 to 24 C atoms in the alkyl group,
N-cocoalkyl aminopropionate,
cocoacylaminoethyl aminopropionate
C₁₂-C₁₈ acyl sarcosine,
N-alkyl-N,N-dimethylammonium glycinates, for example cocoalkyl-dimethylammonium glycinate,
N-acylaminopropyl-N,N-dimethylammonium glycinates, for example cocoacylaminopropyldimethylammonium glycinate,
2-alkyl-3-carboxymethyl-3-hydroxyethylimidazolines with in each case 8 to 18 C atoms in the alkyl or acyl group
cocoacylaminoethylhydroxyethylcarboxymethyl glycinate
the compounds known by the INCI name Cocamidopropyl Betaine,
the compounds known by the INCI name Disodium Cocoamphodiacetates
   wherein preferred agents include the amphoteric surfactant(s) in quantities of 0.5 to 9 wt. %, preferably of 0.75 to 8 wt. % and in particular of 1 to 7.5 wt. %, in each case relative to the entire agent.

More preferred cosmetic agents include betaines of formula (Bet-I) as amphoteric surfactants

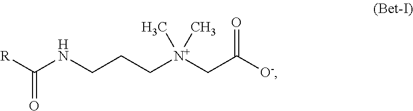

in which R denotes a straight-chain or branched, saturated or mono- or polyunsaturated alkyl or alkenyl residue with 8 to 24 carbon atoms.

According to NCI nomenclature, these surfactants are designated Amidopropylbetaines, wherein those representatives which are derived from coconut fatty acids are preferred and are designated Cocamidopropylbetaines. Surfactants of formula (Bet-I) which are more preferably used according to the invention are those which are a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻

Surfactants of formula (Bet-I) are more preferably used within relatively narrow quantity ranges. Preferred agents according to the invention are here those which, relative to the weight thereof, include 0.25 to 8 wt. %, further preferably 0.5 to 7 wt. %, further preferably 0.75 to 6.5 wt. % and in particular 1 to 5.5 wt. % of surfactant(s) of formula (Bet-I).

In addition to or instead of the amphoteric surfactant(s) of formula (Bet-I), the cosmetic agents according to the invention may particularly preferentially include betaines of formula (Bet-II) as amphoteric surfactants

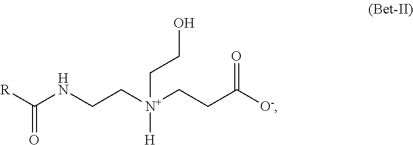

in which R denotes a straight-chain or branched, saturated or mono- or polyunsaturated alkyl or alkenyl residue with 8 to 24 carbon atoms.

According to INCI nomenclature, these surfactants are designated Amphoacetates, wherein those representatives which are derived from coconut fatty acids are preferred and are designated Cocoamphoacetates.

For manufacturing reasons, surfactants of this type always also include betaines of formula (Bet-IIa)

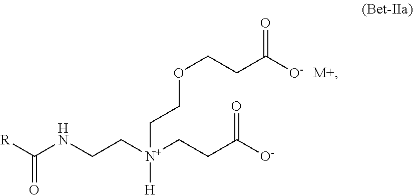

in which R denotes a straight-chain or branched, saturated or mono- or polyunsaturated alkyl or alkenyl residue with 8 to 24 carbon atoms and M denotes a cation. According to INCI nomenclature, these surfactants are designated Amphodiacetates, wherein those representatives which are derived from coconut fatty acids are preferred and are designated Cocoamphodiacetates.

Surfactants of formula (Bet-II) which are more preferably used according to the invention are those which are a mixture of the following representatives:

H₃C—(CH₂)₇—C(O)—NH—(CH₂)₂NH⁺(CH₂CH₂OH)CH₂CH₂COO⁻

H₃C—(CH₂)₉—C(O)—NH—(CH₂)₂NH⁺(CH₂CH₂OH)CH₂CH₂COO⁻

H₃C—(CH₂)₁₁—C(O)—NH—(CH₂)₂NH⁺(CH₂CH₂OH)CH₂CH₂COO⁻

H₃C—(CH₂)₁₃—C(O)—NH—(CH₂)₂NH⁺(CH₂CH₂OH)CH₂CH₂COO⁻

H₃C—(CH₂)₁₅—C(O)—NH—(CH₂)₂NH⁺(CH₂CH₂OH)CH₂CH₂COO⁻

H₃C—(CH₂)₇—CH═CH—(CH₂)₇—C(O)—NH—(CH₂)₂NH⁺(CH₂CH₂OH)CH₂CH₂COO⁻

Surfactants of formula (Bet-II) are more preferably used within relatively narrow quantity ranges. Preferred agents according to the invention are here those which, relative to the weight thereof, include 0.25 to 8 wt. %, further preferably 0.5 to 7 wt. %, further preferably 0.75 to 6.5 wt. % and in particular 1 to 5.5 wt. % of surfactant(s) of formula (Bet-II).

To summarize, preferred cosmetic agents according to the invention are those in which the residue R in the formulae (Bet-I) and (Bet-II) is selected from H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇— or mixtures of these.

More preferred nonionic surfactants are alkyl polyglycosides. Preferred cosmetic agents according to the invention are therefore those which, relative to the weight thereof, include as nonionic surfactants 0.1 to 20 wt. % of alkyl polyglycosides of general formula RO—(Z)ₓ, wherein R denotes alkyl, Z denotes sugar and x denotes the number of sugar units.

Alkyl polyglycosides which are preferred according to the invention are those which correspond to general formula RO—(Z)ₓ, wherein R denotes alkyl, Z denotes sugar and x denotes the number of sugar units. More preferred such alkyl polyglycosides are those in which R
substantially consists of C₈ and Cm alkyl groups,
substantially consists of C₁₂ and C₁₄ alkyl groups,
substantially consists of C₈ to C₁₆ alkyl groups or
substantially consists of C₁₂ to C₁₆ alkyl groups or
substantially consists of C₁₆ to C₁₈ alkyl groups.

Any desired mono- or oligosaccharides may be used as the sugar building block Z. Sugars with 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is more preferred.

Alkyl polyglycosides usable according to the invention include on average 1.1 to 5 sugar units. Alkyl polyglycosides with x values of 1.1 to 2.0 are preferred. Alkyl glycosides in which x is 1.1 to 1.8 are particularly preferred.

Further surfactants which, in particular mixed with alkyl polyglycosides, may more advantageously be used in the agents according to the invention are glutamates, aspartates and sulfoacetates. Preferred cosmetic agents according to the invention are here those which, relative to the weight thereof, include 0.1 to 20 wt. % of fatty acid glutamates (acyl glutamates) and/or fatty acid aspartates (acyl aspartates) and/or alkyl sulfoacetates (sulfoacetic acid alkyl esters).

Acyl glutamates can be described by the formula

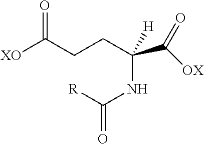

in which R—CO denotes a linear or branched acyl residue with 6 to 22 carbon atoms and 0 and/or 1, 2 or 3 double bonds and X denotes hydrogen, an alkali metal and/or alkaline earth metal, ammonium, alkylammonium, alkanolammonium or glucammonium.

It has surprisingly been found that blends of alkyl glucosides with acyl glutamates have very good dermatological compatibility and improved foaming capacity with regard both to the basic foam and to foam stability in the presence of water hardness.

Typical examples of suitable acyl glutamates are anionic surfactants which are derived from fatty acids with 6 to 22, preferably 12 to 18 carbon atoms, such as for example C_{12/14} or C_{12/18} coconut fatty acid, lauric acid, myristic acid, palmitic acid and/or stearic acid. Sodium N-cocoyl L-glutamate and sodium N-stearoyl L-glutamate are more preferred.

The agents according to the invention may include the alkyl and/or alkenyl oligoglucosides and the acyl glutamates in a weight ratio of 1:99 to 99:1, preferably 10:90 to 90:10 and in particular 80:20 to 50:50. Acyl aspartates can be described by the formula

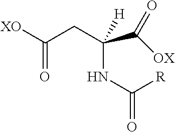

in which R—CO denotes a linear or branched acyl residue with 6 to 22 carbon atoms and 0 and/or 1, 2 or 3 double bonds and X denotes hydrogen, an alkali metal and/or alkaline earth metal, ammonium, alkylammonium, alkanolammonium or glucammonium.

Typical examples of suitable acyl aspartates are anionic surfactants which are derived from fatty acids with 6 to 22, preferably 12 to 18 carbon atoms, such as for example C_{12/14} or C_{12/18} coconut fatty acid, lauric acid, myristic acid, palmitic acid and/or stearic acid. Sodium N-cocoyl L-aspartate and sodium N-stearoyl L-aspartate are more preferred.

The agents according to the invention may include the alkyl and/or alkenyl oligoglucosides and the acyl aspartates likewise in a weight ratio of 1:99 to 99:1, preferably 10:90 to 90:10 and in particular 80:20 to 50:50.

Sulfoacetates (sulfoacetic acid esters) are conventionally salts of esters of sulfoacetic acid and may be described by the general formula

R—O—C(O)—CH₂—SO₂—OX

in which R denotes a linear or branched alkyl or alkenyl residue with 6 to 22 carbon atoms and 0 and/or 1, 2 or 3 double bonds and X denotes hydrogen, an alkali metal and/or alkaline earth metal, ammonium, alkylammonium, alkanolammonium or glucammonium.

It is more preferred to use the sulfoacetic acid sodium salt with the INCI name: Sodium Lauryl Sulfoacetate:

H₃C—(CH₂)₁₁—O—CO—CH₂—SO₂—ONa

Sodium lauryl sulfoacetate is a white, free-flowing powder, which gives a neutral reaction and has good foaming capacity, wetting capacity and dispersing power.

Cationic surfactants of the type including quaternary ammonium compounds, esterquats and amidoamines may be used according to the invention. Preferred quaternary ammonium compounds are ammonium halides, in particular chlorides and bromides, such as alkyltrimethylammonium chlorides, dialkyldimethylammonium chlorides and trialkylmethylammonium chlorides. The long alkyl chains of these surfactants preferably comprise 10 to 18 carbon atoms, such as for example in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are the imidazolium compounds known under the INCI names Quaternium-27 and Quaternium-83.

More preferred hair cleaning agents are characterized in that, relative to the weight thereof, they include as cationic conditioning substance 0.05 to 7.5 wt. %, preferably 0.1 to 5 wt. %, more preferably 0.2 to 3.5 wt. % and in particular 0.25 to 2.5 wt. % of cationic surfactant(s) from the group of quaternary ammonium compounds and/or esterquats and/or amidoamines, wherein preferred cationic surfactant(s) is/are selected from
alkyltrimethylammonium chlorides with preferably 10 to 18 C atoms in the alkyl residue and/or
dialkyldimethylammonium chlorides with preferably 10 to 18 C atoms in the alkyl residue and/or
trialkylmethylammonium chlorides with preferably 10 to 18 C atoms in the alkyl residue and/or
cetyltrimethylammonium chloride and/or
stearyltrimethylammonium chloride and/or
distearyldimethylammonium chloride and/or
lauryldimethylammonium chloride and/or
lauryldimethylbenzylammonium chloride and/or
tricetylmethylammonium chloride
Quaternium-27 and/or
Quaternium-83 and/or
N-methyl-N(2-hydroxyethyl)-N,N-(ditallowacyloxyethyl)ammonium methosulfate and/or
N-methyl-N(2-hydroxyethyl)-N,N-(distearoyloxyethyl)ammonium methosulfate and/or
N,N-dimethyl-N,N-distearoyloxyethylammonium chloride and/or
N,N-di-(2-hydroxyethyl)-N,N-(fatty acid ester ethyl)ammonium chloride.

The conditioning effects of the agents according to the invention may be still further enhanced by using specific conditioning substances. The latter are preferably selected from specific groups of per se known conditioning substances, since, with regard to formulation and conditioning effect, said conditioning substances go together outstandingly well with the silicones used according to the invention.

Cosmetic agents which are preferred according to the invention are characterized in that, relative to the weight thereof, they additionally include conditioning substance(s) in quantities of 0.001 to 10 wt. %, preferably 0.005 to 7.5 wt. %, more preferably 0.01 to 5 wt. % and in particular 0.05 to 2.5 wt. %, wherein preferred conditioning substance(s) are selected from the group
i. L-carnitine and/or the salts thereof;
ii. panthenol and/or pantothenic acid;
iii. 2-furanones and/or the derivatives thereof, in particular pantolactone;
iv. taurine and/or the salts thereof;
v. niacinamide;
vi. ubiquinone
vii. ectoine;
viii. allantoin.

In hair treatment agents according to the invention of this embodiment, the silicones used according to the invention are combined with at least one conditioning substance which is selected from L-carnitine, and/or the salts thereof, panthenol and/or pantothenic acid, 2-furanones and/or the derivatives thereof, in particular pantolactone, taurine and/or the salts thereof, niacinamide, ubiquinones, ectoine, allantoin. These conditioning substances are described below:
L-Camitine (IUPAC name (R)-(3-carboxy-2-hydroxypropyl)-N,N,N-trimethylammonium hydroxide) is a naturally occurring, vitamin-like substance which plays an essential role in the energy metabolism of human, animal and plant cells. L-Carnitine may be obtained on an industrial scale via various pathways. For example via a biotechnological process which imitates physiological biosynthesis: in large fermentation tanks, the precursor of L-carnitine (γ-butyrobetaine) is converted with the assistance of Gram-negative bacteria (rhizobia) into L-carnitine.

As betaine L-Carnitine is capable of forming addition compounds and double salts. L-Carnitine derivatives which are preferred according to the invention are in particular selected from acetyl-L-carnitine, L-carnitine fumarate, L-carnitine citrate, lauroyl-L-carnitine, and more preferably L-carnitine tartrate. The stated L-carnitine compounds are obtainable for example from Lonza GmbH (Wuppertal, Germany).

Cosmetic agents which are preferred according to the invention are characterized in that, relative to the weight thereof, they include 0.001 to 10 wt. %, preferably 0.005 to 7.5 wt. %, more preferably 0.01 to 5 wt. % and in particular 0.05 to 2.5 wt. % of L-camitine or L-carnitine derivatives, wherein preferred L-carnitine derivatives are selected from acetyl-L-camitine, L-carnitine fumarate, L-camitine citrate, lauroyl-L-carnitine and in particular L-carnitine tartrate.

Panthenol (IUPAC name: (+)-(R)-2,4-dihydroxy-N-(3-hydroxypropyl)-3,3-dimethylbutyramide) is converted in the body into pantothenic acid. Pantothenic acid is a vitamin from the B vitamin group (vitamin B₅).

Cosmetic agents which are preferred according to the invention are characterized in that, relative to the weight thereof, they include 0.01 to 5 wt. %, preferably 0.05 to 2.5 wt. %, more preferably 0.1 to 1.5 wt. % and in particular 0.25 to 1 wt. % of panthenol ((±)-2,4-dihydroxy-N-(3-hydroxypropyl)-3,3-dimethylbutyramide).

A further, more preferably usable conditioning substance which has activating properties is taurine. Cosmetic agents which are preferred according to the invention include, relative to the weight thereof, 0.01 to 15 wt. %, preferably 0.025 to 12.5 wt. %, more preferably 0.05 to 10 wt. %, further preferably 0.1 to 7.5 wt. % and in particular 0.5 to 5 wt. % of taurine (2-aminoethanesulfonic acid).

A further preferred group of conditioning substances in the agents according to the invention are vitamins, provitamins or vitamin precursors, which are described below: The group of substances designated vitamin A includes retinol (vitamin A₁) and 3,4-didehydroretinol (vitamin A₂). β-Carotene is the provitamin of retinol. Examples of substances which may be considered according the invention as the vitamin A component are vitamin A acid and the esters thereof, vitamin A aldehyde and vitamin A alcohol and the esters thereof such as the palmitate and the acetate. The agents according to the invention preferably include the vitamin A component in quantities of from 0.05-1 wt. %, relative to the total preparation.

The vitamin B group or the vitamin B complex includes, inter alia
vitamin B₁ (thiamin)
vitamin B₂ (riboflavin)
vitamin B₃. This designation is frequently used for the compounds nicotinic acid and nicotinamide (niacinamide). Nicotinamide is preferred according to the invention and is preferably present in the agents according to the invention in quantities of from 0.05 to 1 wt. %, relative to the total agent.
vitamin B₅ (pantothenic acid, panthenol and pantolactone). In the context of this group, panthenol and/or pantolactone are preferably used. Derivatives of panthenol which may be used according to the invention are in particular the esters and ethers of panthenol and cationically derivatized panthenols. Individual representatives are for example panthenol triacetate, panthenol monoethyl ether and the monoacetate thereof and cationic panthenol derivatives. The stated compounds of the vitamin B₅ type are preferably included in the agents according to the invention in quantities of from 0.05-10 wt. %, relative to the total agent. Quantities of 0.1-5 wt. % are more preferred.
vitamin B₆ (pyridoxine as well as pyridoxamine and pyridoxal).
   Vitamin C (ascorbic acid). Vitamin C is preferably used in the agents according to the invention in quantities of from 0.1 to 3 wt. %, relative to the total agent. Use in the form of the palmitic acid ester, the glucosides or phosphates may be preferred. Use in combination with tocopherols may likewise be preferred.
   Vitamin E (tocopherols, in particular α-tocopherol). Tocopherol and the derivatives thereof, which include in particular the esters such as the acetate, the nicotinate, the phosphate and the succinate, are preferably present in the agents according to the invention in quantities of from 0.054 wt. %, relative to the total agent.
   Vitamin F. The term “vitamin F” is conventionally understood to mean essential fatty acids, in particular linoleic acid, linolenic acid and arachidonic acid.
   Vitamin H. Vitamin H denotes the compound (3aS,4S,6aR)-2-oxohexahydrothienol[3,4-d]-imidazole-4-valeric acid, which is now, however, known by the common name biotin. Biotin is present in the agents according to the invention preferably in quantities of from 0.0001 to 1.0 wt. %, in particular in quantities of from 0.001 to 0.01 wt. %.

To summarize, preferred cosmetic agents according to the invention are those which, relative to the weight thereof, include 0.1 to 5 wt. %, preferably 0.2 to 4 wt. %, more preferably 0.25 to 3.5 wt. %, further preferably 0.5 to 3 wt. % and in particular 0.5 to 2.5 wt. % of vitamins and/or provitamins and/or vitamin precursors which are preferably assigned to groups A, B, C, E, F and H, wherein preferred agents include 2,4-dihydroxy-N-(3-hydroxypropyl)-3,3-dimethylbutyramide, provitamin B₅) and/or pantothenic acid (vitamin B₃, vitamin B₅) and/or niacin, niacinamide or nicotinamide (vitamin B₃) and/or L-ascorbic acid, (vitamin C) and/or thiamin (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂.

It has been found that specific quinones are particularly suitable as a conditioning substance. More preferred cosmetic agents according to the invention are characterized in that, relative to the weight thereof, they include as conditioning substance 0.0001 to 1 wt. %, preferably 0.001 to 0.5 wt. % and more preferably 0.005 to 0.1 wt. % of at least one ubiquinone and/or at least one ubiquinol and/or at least one derivative of these substances, wherein preferred agents include a ubiquinone of formula (Ubi)

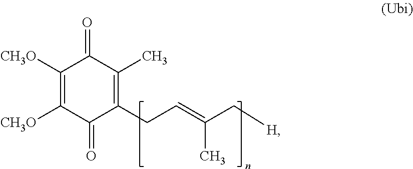

in which n denotes the values 6, 7, 8, 9 or 10, more preferably 10 (coenzyme Q10). Alternatively or in addition to the more preferred ubiquinones, the agents according to the invention may also include plastoquinones. Preferred agents according to the invention are in this case characterized in that they include 0.0002 to 4 wt. %, preferably 0.0005 to 3 wt. %, more preferably 0.001 to 2 wt. %, further preferably 0.0015 to 1 and in particular 0.002 to 0.5 wt. % of at least one plastoquinone of formula (Ubi-b)

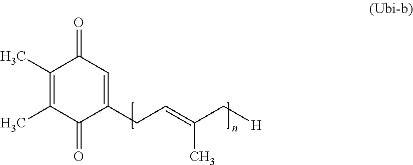

in which n denotes values of 1 to 20, preferably of 2 to 15 and in particular of 5, 6, 7, 8, 9 or 10, wherein agents more preferably include plastoquinone PQ-9.

The agents according to the invention may include ectoine as a further conditioning enhancer. Ectoine ((4S)-2-methyl-1,4,5,6-tetrahydropyrimidine-4-carboxylic acid) is a natural substance which belongs to the group of compatible solutes. This low molecular weight organic compound which has a strong water-binding action occurs in halophilic bacteria and enables these extremophilic organisms to survive under stress conditions. Cosmetic agents which are preferred according to the invention are characterized in that, relative to the weight thereof, they include 0.001 to 10 wt. %, preferably 0.01 to 5 wt. %, more preferably 0.05 to 2.5 wt. % and in particular 0.1 to 1 wt. % of (S)-2-methyl-1,4,5,6-tetrahydro-4-pyrimidinecarboxylic acid (ectoine) and the physiologically acceptable salts of this compound and/or (S,S)-5-hydroxy-2-methyl-1,4,5,6-tetrahydro-4-pyrimidinecarboxylic acid (hydroxyectoine) and the physiologically acceptable salts of this compound.

Allantoin is a further conditioning substance. In various animal species, especially in mammals, allantoin (5-ureidohydantoin, N-(2,5-dioxo-4-imidazolidinyl)urea) is, in addition to uric acid, the final product of the breakdown of nucleic acids, specifically of purine bases.

Allantoin is used in cosmetics in skin creams, sunscreen agents, shaving lotions, in tooth cream and in agents to counter excessive sweating (hyperhidrosis) and skin irritation. It brings about an acceleration in cell building, cell formation or cell regeneration and soothes the skin. Allantoin also assists healing of slow-healing wounds, but has no antiseptic properties.

Cosmetic agents which are more preferred according to the invention include, relative to the weight thereof, 0.001 to 10 wt. %, preferably 0.01 to 5 wt. %, more preferably 0.05 to 2.5 wt. % and in particular 0.1 to 1 wt. % of 5-ureidohydantoin (allantoin).

The agents according to the invention may include purine and/or purine derivatives as conditioning substances in order to improve the elasticity and strengthen the internal structure of the hair treated with agents according to the invention. In particular the combination of purine and/or purine derivatives with ubiquinones and/or plastoquinones as the conditioning substance results in hair treated with corresponding agents exhibiting inter alia higher measured values in differential thermal analysis and improved wet and dry combability.

Purine (7H-imidazole[4,5-d]pyrimidine) does not occur freely in nature, but forms the parent substance of purines. Purines are in turn a group of important compounds which occur widely in nature and participate in human, animal, plant and microbial metabolic processes and are derived from the parent substance by substitution with OH, NH₂, SH in positions 2, 6 and 8 and/or with CH₃ in positions 1, 3 or 7. Purine may for example be produced from aminoacetonitrile and formamide. Purines and purine derivatives are often isolated from natural substances, but can also be obtained synthetically via many pathways.

Preferred agents according to the invention include purine and/or purine derivatives in relatively narrow quantity ranges. Cosmetic agents which are preferred according to the invention are here characterized in that, relative to the weight thereof, they include 0.001 to 2.5 wt. %, preferably 0.0025 to 1 wt. %, more preferably 0.005 to 0.5 wt. % and in particular 0.01 to 0.1 wt. % of purine(s) and/or purine derivative(s).

Among purine, purines and purine derivatives, some representatives are more preferred according to the invention. Cosmetic agents which are preferred according to the invention are characterized in that, relative to the weight thereof, they include as conditioning substance 0.001 to 2.5 wt. %, preferably 0.0025 to 1 wt. %, more preferably 0.005 to 0.5 wt. % and in particular 0.01 to 0.1 wt. % of purine(s) and/or purine derivative(s), wherein preferred agents include purine and/or purine derivative(s) of formula (Pur-I)

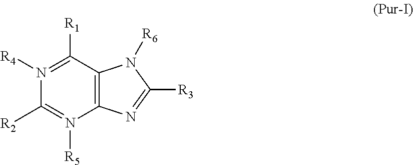

in which the residues R¹, R² and R³ are mutually independently selected from —H, —OH, —NH₂, —SH and the residues R⁴, R⁵ and R⁶ are mutually independently selected from —H, —CH₃ and —CH₂—CH₃, wherein the following compounds are preferred:
purine (R¹═R²═R³═R⁴═R⁵═R⁶═H)
adenine (R¹═NH₂, R²═R³═R⁴═R⁵═R⁶═H)
guanine (R¹═OH, R²═NH₂, R³═R⁴═R⁵═R⁶═H)
uric acid (R¹═R²═R³═OH, R⁴═R⁵═R⁶═H)
hypoxanthine (R¹═OH, R²═R³═R⁴═R⁵═R⁶═H)
6-purinethiol (R¹═SH, R²═R³═R⁴═R⁵═R⁶═H)
6-thioguanine (R¹═SH, R²═NH₂, R³═R⁴═R⁵═R⁶═H)
xanthine (R¹═R²═OH, R³═R⁴═R⁵═R⁶═H)
caffeine (R¹═R²═OH, R³═H, R⁴═R⁵═R⁶═CH₃)
theobromine (R¹═R²═OH, R³═R⁴═H, R⁵═R⁶═CH₃)
theophylline (R¹═R²═OH, R³═H, R⁴═CH₃, R⁵═CH₃, R⁶═H).

It is furthermore advantageous to use purine or purine derivatives and bioquinones in a specific ratio relative to one another. Agents which are preferred according to the invention are here those in which the weight ratio of purine (derivative(s)) and bioquinone(s) amounts to 10:1 to 1:100, preferably 5:1 to 1:50, more preferably 2:1 to 1:20 and in particular 1:1 to 1:10.

As has already been mentioned, caffeine is a more preferred purine derivative, while coenzyme Q10 is a particularly preferred bioquinone. More preferred agents according to the invention are therefore characterized in that, relative to the weight thereof, they include 0.001 to 2.5 wt. %, preferably 0.0025 to 1 wt. %, more preferably 0.005 to 0.5 wt. % and in particular 0.01 to 0.1 wt. % of caffeine and 0.0002 to 4 wt. %, preferably 0.0005 to 3 wt. %, more preferably 0.001 to 2 wt. %, further preferably 0.0015 to 1 and in particular 0.002 to 0.5 wt. % of coenzyme Q10.

The agents according to the invention may also include flavonoids as a conditioning substance. Flavonoids are a group of water-soluble plant dyes and play an important role in the metabolism of many plants. Like phenolic acids, they belong to the class of polyphenols. Far more than 6500 different flavonoids are known which can be divided into flavonols, flavones, flavanones, isoflavonoids and anthocyans.

Flavonoids from any of the six groups may be used according to the invention, wherein specific representatives from the individual groups are preferred as a conditioning substance due to their particularly intense action. Preferred flavonols are quercetin, rutin, kaempferol, myricetin or isorhamnetin, preferred flavanones are catechin, gallocatechin, epicatechin, epigallocatechin gallate, theaflavin or thearubigin, preferred flavones are luteolin, apigenin or morin, preferred flavanones are hesperetin, naringenin or eriodictyol, preferred isoflavonoids are genistein or daidzein, and preferred anthocyanidins (anthocyans) are cyanidin, delphinidin, malvidin, pelargonidin, peonidin or petunidin.

Cosmetic agents which are more preferred according to the invention are characterized in that, relative to the weight thereof, they include 0.001 to 2.5 wt. %, preferably 0.0025 to 1 wt. %, more preferably 0.005 to 0.5 wt. % and in particular 0.01 to 0.1 wt. % of flavonoids, in particular flavonols, more preferably 3,3′,4′,5,7-pentahydroxyflavone (quercetin) and/or 3,3′,4′,5,7-pentahydroxyflavone-3-O-rutinoside (rutin).

It is also preferred to use bisabolol and/or bisabolol oxides as a conditioning substance in the agents according to the invention. Preferred cosmetic agents according to the invention are here those which additionally include 0.001 to 5 wt. %, preferably 0.01 to 4 wt. %, more preferably 0.02 to 2.5 wt. % and in particular 0.1 to 1.5 wt. % of bisabolol and/or oxides of bisabolol, preferably (−)-alpha bisabolol.

Creatine is also suitable according to the invention as a conditioning substance. Creatine (3-methylguanidinoacetic acid) is an organic acid which in vertebrates is involved inter alia with supplying muscles with energy. Creatine is synthesized in the kidneys, the liver and the pancreas. It is derived in formal terms from the amino acids glycine and arginine and 95% of it is present in skeletal muscle. More preferred cosmetic agents according to the invention include, relative to the weight thereof, 0.01 to 15 wt. %, preferably 0.025 to 12.5 wt. %, more preferably 0.05 to 10 wt. %, further preferably 0.1 to 7.5 wt. % and in particular 0.5 to 5 wt. % of N-methylguanidinoacetic acid (creatine).

In addition to the above-stated ingredients and optional further ingredients, the agents according to the invention may include further substances which prevent, alleviate or remedy hair loss. A content of active substances which stabilize the hair root is in particular advantageous. These substances are described below:

Propecia (finasteride) is at present the only preparation which is authorized worldwide and which has been demonstrated in numerous studies to be effective and well tolerated. Propecia ensures that less DHT can be formed from testosterone.
Minoxidil, with or without complementary additives, is probably the oldest hair restorer with proven efficacy. It may only be used externally for treating hair loss. There are hair lotions which include 2%-5% minoxidil and gels with up to 15% minoxidil. Action increases with dosage, but in hair lotions minoxidil is only soluble in a proportion of up to 5%. In many countries, hair lotions with a minoxidil content of up to 2% are obtainable without prescription.
Spironolactone in the form of a hair lotion and in combination with minoxidil may be used for external application to combat hormonal influences on hair follicles.
Spironolactone acts as an androgen receptor blocker, i.e. DHT is prevented from binding to the hair follicles.

To summarize, preferred cosmetic agents according to the invention are those which, relative to the weight thereof, additionally include 0.001 to 5 wt. % of hair root-stabilizing substances, in particular minoxidil and/or finasteride and/or ketoconazole.

Additional antidandruff active ingredients (for example climbazole, piroctone olamine or zinc pyrithione) bring about a targeted reduction in the quantity of the yeast fungus which causes dandruff, returning the microbial flora to its normal percentage composition and reducing desquamation to its physiological level. Laboratory tests have, however, revealed that the various varieties of Pityrosporum ovale respond differently well to the antidandruff active substances. A combination of antidandruff active substances is therefore most successful for achieving the best possible action against all dandruff pathogens.

To summarize, preferred cosmetic agents according to the invention are those which, relative to the weight thereof, additionally include 0.001 to 5 wt. % of antidandruff active substances, in particular piroctone olamine (1-hydroxy-4-methyl-6-(2,4,4-trimethylpentyl)pyridin-2(1H)-one, compound with 2-aminoethanol, 1:1) and/or zinc pyrithione and/or selenium sulfide and/or climbazole and/or salicylic acid or fumaric acid.

In addition to the conditioning substances, the agents according to the invention may include further conditioning substances. Their presence is not absolutely essential for achieving the effect according to the invention, but using these conditioning substances may give rise to further effects, such as a pleasant handle or pleasant tactile properties on application.

The agents according to the invention may more preferentially include one or more amino acids as a further ingredient. Amino acids which are more preferably usable according to the invention originate from the group glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliine), L-trans-4-hydroxyproline, L-5-oxoproline (L-pyroglutamic acid), L-phosphoserine, creatine, 3-methyl-L-histidine, L-ornithine, wherein both the individual amino acids and mixtures may be used.

Preferred agents according to the invention include one or more amino acids in relatively narrow quantity ranges. Cosmetic agents which are preferred according to the invention are here characterized in that, relative to the weight thereof, they include as conditioning substance 0.01 to 5 wt. %, preferably 0.02 to 2.5 wt. %, more preferably 0.05 to 1.5 wt. %, further preferably 0.075 to 1 wt. % and in particular 0.1 to 0.25 wt. % of amino acid(s), preferably from the group glycine and/or alanine and/or valine and/or lysine and/or leucine and/or threonine.

The agents according to the invention may include at least one carbohydrate from the group of monosaccharides, disaccharides and/or oligosaccharides as a further component. Cosmetic agents which are preferred according to the invention are here characterized in that, relative to the weight thereof, they include as conditioning substance 0.01 to 5 wt. %, preferably 0.05 to 4.5 wt. %, more preferably 0.1 to 4 wt. %, further preferably 0.5 to 3.5 wt. % and in particular 0.75 to 2.5 wt. % of carbohydrate(s) selected from monosaccharides, disaccharides and/or oligosaccharides, wherein preferred carbohydrates are selected from monosaccharides, in particular D-ribose and/or D-xylose and/or L-arabinose and/or D-glucose and/or D-mannose and/or D-galactose and/or D-fructose and/or sorbose and/or L-fucose and/or L-rhamnose, disaccharides, in particular sucrose and/or maltose and/or lactose and/or trehalose and/or cellobiose and/or gentiobiose and/or isomaltose.

More preferred agents according to the invention include, relative to the weight thereof,
0.005 to 0.015 wt. % of caffeine and 0.75 to 1.5 wt. % of glucose monohydrate,
0.005 to 0.015 wt. % of caffeine and 0.75 to 1.5 wt. % of sucrose,
0.005 to 0.015 wt. % of caffeine and 0.75 to 1.5 wt. % of fructose.

As has already been mentioned, agents according to the invention preferably include (an) amino acid(s). Amino acids which are more preferably usable according to the invention originate from the group glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliine), L-trans-4-hydroxyproline, L-5-oxoproline (L-pyroglutamic acid), L-phosphoserine, creatine, 3-methyl-L-histidine, L-ornithine, wherein both the individual amino acids and mixtures may be used.

Preferred agents according to the invention include one or more amino acids in relatively narrow quantity ranges. Cosmetic agents which are preferred according to the invention are here characterized in that they additionally include 0.05 to 5 wt. %, preferably 0.1 to 2.5 wt. %, more preferably 0.15 to 1 wt. % and in particular 0.2 to 0.5 wt. % of amino acid(s), preferably (an) amino acid(s) from the group glycine and/or alanine and/or valine and/or lysine and/or leucine and/or threonine.

It goes without saying that the silicones used according to the invention may be used together with further conventional silicones.

Agents which are preferred according to the invention are characterized in that they include at least one further silicone, preferably a silicone which is selected from:
(i) polyalkylsiloxanes, polyarylsiloxanes, polyalkylarylsiloxanes, which are volatile or non-volatile, straight-chain, branched or cyclic, crosslinked or uncrosslinked;
(ii) polysiloxanes which include in their general structure one or more organofunctional groups which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)/polyoxyalkylene(B) block copolymers of the type (A-B)ₙ with n>3;
(iv) grafted silicone polymers with an organic parent structure including no silicone, which polymers consist of an organic main chain which is formed from organic monomers including no silicone and onto which at least one polysiloxane macromer has been grafted in the chain and optionally onto at least one chain end;
(v) grafted silicone polymers with a polysiloxane parent structure, onto which organic monomers including no silicone have been grafted, which silicone polymers have a polysiloxane main chain onto which at least one organic macromer has been grafted in the chain and optionally onto at least one of the ends thereof;
   or mixtures thereof.

Agents which are more preferred according to the invention include the further silicone(s) preferably in quantities of 0.1 to 10 wt. %, preferably of 0.25 to 7 wt. % and in particular of 0.5 to 5 wt. %, in each case relative to the entire agent.

Preferred silicones are described hereinafter.

More preferred agents according to the invention are characterized in that they include at least one amino-functional silicone of formula Si-I

(CH₃)₃Si-[O—Si(CH₃)₂]ₓ—O—Si(CH₃)₃ (Si-I),

in which x denotes a number from 0 to 100, preferably from 0 to 50, further preferably from 0 to 20 and in particular from 0 to 10.

According to INCI nomenclature, these silicones are known as Dimethicones. Silicones of formula Si-I which are preferably used for the purposes of the present invention are the compounds:

(CH₃)₃Si—O—Si(CH₃)₃, (CH₃)₃Si—O—(CH₃)₂Si—O—Si(CH₃)₃, (CH₃)₃Si—[O—(CH₃)₂Si]₂—O—Si(CH₃)₃, (CH₃)₃Si-[O—(CH₃)₂Si]₃—O—Si(CH₃)₃, (CH₃)₃Si-[O—(CH₃)₂Si]₄—O—Si(CH₃)₃, (CH₃)₃Si-[O—(CH₃)₂Si]₅—O—Si(CH₃)₃, (CH₃)₃Si-[O—(CH₃)₂Si]₆—O—Si(CH₃)₃, (CH₃)₃Si-[O—(CH₃)₂Si]₇—O—Si(CH₃)₃, (CH₃)₃Si-[O—(CH₃)₂Si]₈—O—Si(CH₃)₃, (CH₃)₃Si-[O(CH₃)₂Si]₉—O—Si(CH₃)₃, (CH₃)₃Si-[O⁻(CH₃)₂Si]₁₀—O—Si(CH₃)₃, (CH₃)₃Si-[O(CH₃)₂Si]₁₁—O—Si(CH₃)₃, (CH₃)₃Si-[O—(CH₃)₂Si]₁₂—O—Si(CH₃)₃, (CH₃)₃Si-[O—(CH₃)₂Si]₁₃—O—Si(CH₃)₃, (CH₃)₃Si-[O—(CH₃)₂Si]₁₄—O—Si(CH₃)₃, (CH₃)₃Si-[O—(CH₃)₂Si]₁₅—O—Si(CH₃)₃, (CH₃)₃Si—[O—(CH₃)₂Si]₁₆—O—Si(CH₃)₃, (CH₃)₃Si-[O—(CH₃)₂Si]₁₇—O—Si(CH₃)₃, (CH₃)₃Si-[O—(CH₃)₂Si]₁₈—O—Si(CH₃)₃, (CH₃)₃Si-[O—(CH₃)₂Si]₁₉—O—Si(CH₃)₃, (CH₃)₃Si-[O⁻(CH₃)₂Si]₂₀—O—Si(CH₃)₃, wherein (CH₃)₃Si—O—Si(CH₃)₃, (CH₃)₃Si—O—(CH₃)₂Si—O—Si(CH₃)₃ and/or (CH₃)₃Si-[O—(CH₃)₂Si]₂—O—Si(CH₃)₃ are more preferred.

Mixtures of the above-stated silicones may of course be present in agents according to the invention.

Silicones which are preferably usable according to the invention exhibit viscosities at 20° C. of 0.2 to 2 mm² s⁻¹, wherein silicones with viscosities of 0.5 to 1 mm² s⁻¹ are more preferred.

More preferred agents according to the invention include one or more amino-functional silicones. Such silicones may, for example, be described by the formula

M(RₐQ_{b}SiO_{(4-a-b)/2})ₓ(R_{c}SiO_{(4-c)/2})_{y}M

wherein in the above formula R is a hydrocarbon or a hydrocarbon residue with 1 to approximately 6 carbon atoms, Q is a polar residue of the general formula —R¹HZ, in which R¹ is a divalent linking group, which is attached to hydrogen and the residue Z and is composed of carbon and hydrogen atoms, carbon, hydrogen and oxygen atoms or carbon, hydrogen and nitrogen atoms, and Z is an organic, amino-functional residue, which includes at least one amino-functional group; “a” assumes values in the range from approximately 0 to approximately 2, “b” assumes values in the range from approximately 1 to approximately 3, “a”+“b” is less than or equal to 3, and “c” is a number in the range from approximately 1 to approximately 3, and x is a number in the range from 1 to approximately 2000, preferably from approximately 3 to approximately 50 and most preferably from approximately 3 to approximately 25, and y is a number in the range from approximately 20 to approximately 10000, preferably from approximately 125 to approximately 10000 and most preferably from approximately 150 to approximately 1000, and M is a suitable silicone end group, as known in the prior art, preferably trimethylsiloxy. Non-limiting examples of the residues represented by R include alkyl residues, such as methyl, ethyl, propyl, isopropyl, isopropyl, butyl, isobutyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R is preferably an alkyl residue, which includes 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, (CH₂)₃CC(O)OCH₂CH₂—, —C₆H₄C₆H₄—, —C₆H₄CH₂C₆H₄—; and —(CH₂)₃C(O)SCH₂CH₂—. Z is an organic, amino-functional residue including at least one functional amino group. A possible formula for Z is NH(CH₂)_{z}NH₂, in which z is 1 or more. Another possible formula for Z is —NH(CH₂)_{z}(CH₂)_{zz}NH, in which both z and zz are mutually independently 1 or more, wherein this structure comprises diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is independently selected from the group consisting of hydrogen and alkyl groups with 1 to 12 carbon atoms, and zz is 0. Q is most preferably a polar amino-functional residue of formula —CH₂CH₂CH₂NHCH₂CH₂NH₂. In the formulae, “a” assumes values in the range from 0 to approximately 2, “b” assumes values in the range from approximately 2 to approximately 3, “a”+“b” is less than or equal to 3, and “c” is a number in the range from approximately 1 to approximately 3. The molar ratio of the RₐQ_{b} SiO_{(4-a-b)/2} units to the R_{c}SiO_{(4-c)/2} units is in the range from approx. 1:2 to 1:65, preferably from approximately 1:5 to approximately 1:65 and most preferably from approximately 1:15 to approximately 1:20. If one or more silicones of the above formula are used, then the various variable substituents in the above formula may be different in the various silicone components which are present in the silicone mixture.

Preferred agents according to the invention are characterized in that they include an amino-functional silicone of formula (Si-II)

R′ₐG₃₋ₐ-Si(OSiG₂)ₙ-(OSiG_{b}R′_{2-b})ₘ—O—SiG₃₋ₐ-R′ₐ (Si-II),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a denotes a number between 0 and 3, in particular 0;
b denotes a number between 0 and 1, in particular 1,
m and n are numbers, the sum of which (m+n) amounts to between 1 and 2000, preferably between 50 and 150, wherein n preferably assumes values from 0 to 1999 and in particular from 49 to 149 and m preferably assumes values from 1 to 2000, in particular from 1 to 10,
R′ is a monovalent residue selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻, wherein each Q denotes a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
R″ denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

More preferred agents according to the invention are characterized in that they include at least one amino-functional silicone of formula (Si-IIa)

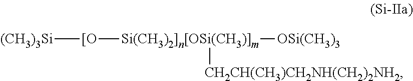

in which m and n are numbers, the sum of which (m+n) amounts to between 1 and 2000, preferably between 50 and 150, wherein n preferably assumes values from 0 to 1999 and in particular from 49 to 149 and m preferably assumes values from 1 to 2000, in particular from 1 to 10.

These silicones are denoted in accordance with the INCI Declaration as Trimethylsilylamodimethicones.

Agents according to the invention which are more preferred are also those which include an amino-functional silicone of formula (Si-IIb)

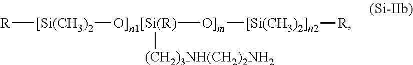

in which R denotes —OH, —O—CH₃ or a —CH₃ group and m, n1 and n2 are numbers the sum of which (m+n1+n2) amounts to between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

These silicones are denoted in accordance with the INCI Declaration as Amodimethicones.

Irrespective of which amino-functional silicones are used, preferred agents according to the invention are those which include an amino-functional silicone, the amine value of which is above 0.25 meq/g, preferably above 0.3 meq/g and in particular above 0.4 meq/g. The amine value here denotes the milliequivalents of amine per gram of the amino-functional silicone. It can be determined by titration and may also be stated in the unit mg of KOH/g.

Agents which are preferred according to the invention are characterized in that, relative to the weight thereof, they include 0.01 to 10 wt. %, preferably 0.1 to 8 wt. %, more preferably 0.25 to 7.5 wt. % and in particular 0.5 to 5 wt. % of amino-functional silicone(s).

The cyclic dimethicones designated according to INCI as Cyclomethicones may preferably be used according to the invention. Preferred agents according to the invention are here those which include at least one silicone of formula Si-III

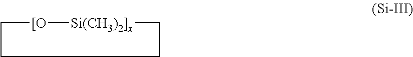

in which x denotes a number from 3 to 200, preferably from 3 to 10, further preferably from 30 to 7 and in particular 3, 4, 5 or 6.

The above-described silicones have a backbone which is made up of Si—O—Si units. These Si—O—Si units may, of course, also be interrupted by carbon chains. Corresponding molecules are obtainable by chain extension reactions and are preferably used in the form of silicone-in-water emulsions.

Agents which are likewise preferred according to the invention are characterized in that they include at least one silicone of formula Si-IV

R³Si-[O—SiR²]ₓ—(CH₂)ₙ-[O—SiR²]_{y}—O—SiR³ (Si-IV),

in which R denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, further preferably from 0 to 7 and in particular 0, 1, 2, 3, 4, 5 or 6, and n denotes a number from 0 to 10, preferably from 1 to 8 and in particular 2, 3, 4, 5, 6.

The silicones are preferably water-soluble. Preferred agents according to the invention are characterized in that they include at least one water-soluble silicone.

The agents according to the invention may include at least one proteolipid of formula (P-I) as a further ingredient

R′—X—R″ (P-I),

in which
R′ denotes a straight-chain or branched, saturated or unsaturated hydrocarbon residue with 11 to 24 carbon atoms,
R″ means a protein, a peptide or a protein hydrolysate,
X denotes —C(O)O— or —N⁺(R^{III}₂)R^{IV}— or —N(R^{III})R^{IV}— or —C(O)—N(R^{V})R^{VI}—,
R^{III} means —(CH₂)ₓ—CH₃ with x=0-22 and
R^{IV} means —CH₂—CH(OH)—CH₂— or —(CH₂)ₓ— with x=0-22;
R^{V} and R^{VI} mutually independently denote —H or —(CH₂)ₓ—CH₃ with x=0-22;
   with the proviso that R″ denotes keratin or a keratin hydrolysate if X denotes —C(O)O—.

The proteolipids are preferably used within specific quantities in the agents according to the invention. Preferred cosmetic agents according to the invention include, relative to the weight thereof, 0.01 to 10 wt. %, preferably 0.02 to 5 wt. %, more preferably 0.05 to 2.5 wt. %, further preferably 0.1 to 1 wt. % and in particular 0.15 to 0.5 wt. % of proteolipid(s). The residue R″ in formula (P-I) denotes a peptide or a protein or a protein hydrolysate. If X═—C(O)O—, R″ is selected from the group keratin or keratin hydrolysate.

Preferred residues R″ are oligopetides which have at least one Glu-Glu-Glu amino acid sequence, wherein the amino group may assume free or protonated form and the carboxy groups may assume free or deprotonated form.

In this and in all the following formulae, the bracketed hydrogen atom of the amino group and likewise the bracketed hydroxyl group of the acid function mean that the groups in question may be present as such (in which case the compound is an oligopeptide with the relevant number of amino acids as shown (in formula 3 above)) or alternatively that the amino acid sequence is present in an oligopeptide which comprises still further amino acids (depending on where the further amino acid(s) is/are attached, the bracketed components of the above-stated formula are replaced by the further amino acid residue(s)).

Oligopeptides for the purposes of the present application are amino acid condensation products linked by acid amide type peptide bonds and which include at least 3 and a maximum of 25 amino acids.

In hair treatment agents of the above-described embodiment which are preferred according to the invention, the oligopeptide (=the residue R″) comprises 5 to 15 amino acids, preferably 6 to 13 amino acids, more preferably 7 to 12 amino acids and in particular 8, 9 or 10 amino acids.

The molar mass of the proteolipid present in the agents according to invention may vary depending on whether further amino acids are attached to the Glu-Glu-Glu sequence and depending on the nature of these amino acids and as a function of the residues R′ and optionally R^{III} and R^{IV} which are selected. Cosmetic agents which are preferred according to the invention are characterized in that the proteolipid has a molar mass of 1000 to 30000 Da, preferably of 1250 to 25000 Da, more preferably of 1500 to 20000 Da and in particular of 2000 to 15000 Da.

Oligopeptides which are preferably used as residue R″ are those which do not solely consist of the three glutamic acids, but instead comprise further amino acids attached to this sequence. These further amino acids are preferably selected from specific amino acids, while certain other representatives are less preferred according to the invention.

It is accordingly preferred for the residue R″ of the proteolipids used in the agents according to the invention to include no methionine. It is further preferred for the residue R″ of the proteolipids used in the agents according to the invention to include no cysteine and/or cystine.

It is further preferred for the residue R″ of the proteolipids used in the agents according to the invention to include no aspartic acid and/or asparagine. It is further preferred for the residue R″ of the proteolipids used in the agents according to the invention to include no serine and/or threonine.

In contrast, it is preferred for the residue R″ of the proteolipids used in the agents according to the invention to include tyrosine. It is further preferred for the residue R″ of the proteolipids used in the agents according to the invention to include leucine. It is further preferred for the residue R″ of the proteolipids used in the agents according to the invention to include isoleucine. It is further preferred for the residue R″ of the proteolipids used in the agents according to the invention to include arginine. It is further preferred for the residue R″ of the proteolipids used in the agents according to the invention to include valine.

Oligopeptides or amino acid sequences present in the preferred oligopeptides which are more preferred as residue R″ are described below:

A more preferred oligopeptide additionally includes tyrosine which is preferably attached via the acid function thereof to the Glu-Glu-Glu sequence. Cosmetic agents which are preferred according to the invention are therefore characterized in that the oligopeptide present as residue R″ in the proteolipids of formula (I) comprises at least one Tyr-Glu-Glu-Glu amino acid sequence, wherein the amino group may assume free or protonated form and the carboxy groups may assume free or deprotonated form.

A further more preferred oligopeptide additionally includes isoleucine which is preferably attached via the acid function thereof to the Glu-Glu-Glu sequence. Cosmetic agents which are preferred according to the invention are therefore characterized in that the oligopeptide present as residue R″ in the proteolipids of formula (I) comprises at least one Glu-Glu-Glu-Ile amino acid sequence, wherein the amino group may assume free or protonated form and the carboxy groups may assume free or deprotonated form.

Oligopeptides which comprise both of the above-stated amino acids (tyrosine and isoleucine) are preferred according to the invention. More preferred hair treatment agents according to the invention are here those in which the oligopeptide present as residue R″ in the proteolipids of formula (I) comprises at least one Tyr-Glu-Glu-Glu-Ile amino acid sequence, wherein the amino group may assume free or protonated form and the carboxy groups may assume free or deprotonated form.

Further preferred oligopeptides additionally include arginine, which is preferably attached to isoleucine. Cosmetic agents which are preferred according to the invention are therefore characterized in that the oligopeptide present as residue R″ in the proteolipids of formula (I) comprises at least one Tyr-Glu-Glu-Glu-Ile-Arg amino acid sequence, wherein the amino groups may assume free or protonated form and the carboxy groups may assume free or deprotonated form.

Still more preferred oligopeptides additionally include valine, which is preferably attached to arginine. Cosmetic agents which are further preferred according to the invention are therefore characterized in that the oligopeptide present as residue R″ in the proteolipids of formula (I) comprises at least one Tyr-Glu-Glu-Glu-Ile-Arg-Val amino acid sequence, wherein the amino groups may assume free or protonated form and the carboxy groups may assume free or deprotonated form.

Still more preferred oligopeptides additionally include leucine, which is preferably attached to valine. Cosmetic agents which are further preferred according to the invention are therefore characterized in that the oligopeptide present as residue R″ in the proteolipids of formula (I) comprises at least one Tyr-Glu-Glu-Glu-Ile-Arg-Val-Leu amino acid sequence, wherein the amino groups may assume free or protonated form and the carboxy groups may assume free or deprotonated form.

More preferred oligopeptides additionally include leucine, which is preferably attached to tyrosine. Cosmetic agents which are further preferred according to the invention are therefore characterized in that the oligopeptide present as residue R″ in the proteolipids of formula (I) comprises at least one Leu-Tyr-Glu-Glu-Glu-Ile-Arg-Val-Leu amino acid sequence, wherein the amino groups may assume free or protonated form and the carboxy groups may assume free or deprotonated form.

To summarize, cosmetic agents according to the invention which are preferred are those which include at least one proteolipid of formula (I), in which R″ comprises at least one Leu-Tyr-Glu-Glu-Glu-Ile-Arg-Val-Leu amino acid sequence, wherein the amino groups may assume free or protonated form and the carboxy groups may assume free or deprotonated form.

As has already been mentioned, R″ is selected from the group keratin or keratin hydrolysate if X═—C(O)O—.

In all other cases, the residue R″ in formula (P4) may denote a peptide or a protein or a protein hydrolysate, wherein protein hydrolysates are preferred. Protein hydrolysates are product mixtures which are obtained by acidically, basically or enzymatically catalyzed degradation of proteins. Protein hydrolysates of both plant and animal origin may be used according to the invention.

Animal protein hydrolysates are for example elastin, collagen, keratin, silk and milk protein hydrolysates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

It is preferred according to the invention to use protein hydrolysates of plant origin, for example soy, almond, rice, pea, potato and wheat protein hydrolysates. Such products are obtainable, for example, under the trademarks Gluadin® (Cognis), DiaMin® (Diamalt), Lexein® (Inolex) and Crotein® (Croda).

Preferably, the residue R″ is selected from keratin or keratin hydrolysates irrespective of the X selected in formula (P-I). Preferred cosmetic agents according to the invention are characterized in that they include at least one proteolipid of formula (P-I), in which R″ denotes keratin or a keratin hydrolysate.

In particular, preferred cosmetic agents according to the invention are those which include at least one proteolipid of formula (P4), in which R^{III} means —CH₃ and R^{IV} denotes —(CH₂)ₓ— with x=0, 1 , 2, 3, 4, 5, 6, 7 or 8.

More preferred cosmetic agents according to the invention are further characterized in that they include at least one proteolipid of formula (I), in which X denotes —N⁺(CH₃)₂—CH₂—CH(OH)—CH₂— and R′ denotes —(CH₂)₁₇—CH₃.

Cosmetic agents according to the invention which are likewise further preferred are characterized in that they include at least one proteolipid of formula (P-I), in which X denotes —C(O)—O— and R′ denotes —(CH₂)₁₇—CH₃.

It has proven advantageous to use protein hydrolysates in addition to the proteolipids. Said protein hydrolysates enhance the action of the proteolipids and in turn have their effects enhanced. The protein hydrolysates have been described in detail further above as residue R″. To summarize, preferred cosmetic agents according to the invention are those which, relative to the weight thereof, additionally include 0.01 to 10 wt. %, preferably 0.05 to 7 wt. %, more preferably 0.1 to 5 wt. %, further preferably 0.25 to 2.5 wt. % and in particular 0.5 to 2.0 wt. % of protein hydrolysate(s), preferably keratin hydrolysate(s).

For esthetic reasons, consumers often prefer “clear” products. Cosmetic agents which are preferred according to the invention are therefore characterized in that they are transparent or translucent.

For the purposes of the present invention, transparent or translucent is taken to mean a composition which has an NTU value of below 100. The NTU (Nephelometric Turbidity Unit) value is a unit used in water treatment for measuring turbidity in liquids. It is the unit which indicates the turbidity of a liquid measured with a calibrated nephelometer.

In a preferred embodiment of the invention, an agent according to the invention may furthermore also include UV filters (I). UV filters to be used according to the invention are not subject to any general restrictions with regard to structure and physical properties. Rather, any UV filters usable in the field of cosmetics whose absorption maximum is in the UVA (315-400 nm), the UVB (280-315 nm) or the UVC (<280 nm) range are suitable. UV filters with an absorption maximum in the UVB range, in particular in the range from approximately 280 to approximately 300 nm, are more preferred.

The UV filters are conventionally present in the agents according to the invention in quantities of 0.1-5 wt. % relative to the total agent. Quantities of 0.4-2.5 wt. % are preferred.

The agents according to the invention may furthermore include a 2-pyrrolidinone-5-carboxylic acid and the derivatives thereof (J). The sodium, potassium, calcium, magnesium or ammonium salts, in which, in addition to hydrogen, the ammonium ion bears one to three C₁ to C₄ alkyl groups, are preferred. The sodium salt is particularly preferred. The quantities used in the agents according to the invention amount to 0.05 to 10 wt. %, relative to the total agent, more preferably 0.1 to 5, and in particular 0.1 to 3 wt. %.

It may additionally prove advantageous for penetration aids and/or swelling agents (M) to be present in the agents according to the invention. Examples of these include urea and urea derivatives, guanidine and the derivatives thereof, arginine and the derivatives thereof, water glass, imidazole and the derivatives thereof, histidine and the derivatives thereof, benzyl alcohol, glycerol, glycol and glycol ethers, propylene glycol and propylene glycol ethers, for example propylene glycol monoethyl ether, carbonates, hydrogencarbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol or 1,4-butanediol.

In a further preferred embodiment, the agents according to the invention may include emulsifiers (F). Emulsifiers bring about the formation of water- or oil-resistant adsorption layers at the phase interface, which protect the dispersed droplets from coalescence and so stabilize the emulsion. Emulsifiers, like surfactants, are thus made up of a hydrophobic and a hydrophilic molecular moiety. Hydrophilic emulsifiers preferably form O/W emulsions while hydrophobic emulsifiers preferably form W/O emulsions. An emulsion is taken to mean a droplet distribution (dispersion) of one liquid in another liquid with the input of energy to create stabilizing phase interfaces by means of surfactants. The selection of these emulsifying surfactants or emulsifiers is here determined on the basis of the substances to be dispersed and the particular external phase and the fineness of the emulsion. Examples of emulsifiers which may be used according to the invention are:
addition products of 4 to 30 mol of ethylene oxide and/or 0 to 5 mol of propylene oxide onto linear fatty alcohols with 8 to 22 C atoms, onto fatty acids with 12 to 22 C atoms and onto alkylphenols with 8 to 15 C atoms in the alkyl group,
C₁₂-C₂₂ fatty acid mono- and diesters of addition products of 1 to 30 mol of ethylene oxide onto polyols with 3 to 6 carbon atoms, in particular onto glycerol,
ethylene oxide and polyglycerol addition products onto methyl glucoside/fatty acid esters, fatty acid alkanolamides and fatty acid glucamides,
C₈-C₂₂ alkyl mono- and oligoglycosides and the ethoxylated analogs thereof, wherein degrees of oligomerization of 1.1 to 5, in particular of 1.2 to 2.0, and glucose as the sugar component are preferred,
mixtures of alkyl (oligo)glucosides and fatty alcohols, for example the commercially obtainable product Montanov®68,
addition products of 5 to 60 mol of ethylene oxide onto castor oil and hardened castor oil,
partial esters of polyols with 3-6 carbon atoms with saturated fatty acids with 8 to 22 C atoms,
sterols. Sterols are taken to be a group of steroids which bear a hydroxyl group on C atom 3 of the steroid skeleton and may be isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, these being known as mycosterols.
phospholipids. These are primarily taken to mean glucose phospholipids which are for example obtained as lecithins, or phosphatidylcholines for example from egg yolk or plant seeds (for example soy beans).
fatty acid esters of sugars and sugar alcohols, such as sorbitol,
polyglycerols and polyglycerol derivatives such as for example polyglycerol poly-12-hydroxystearate (commercial product Dehymuls® PGPH),
linear and branched fatty acids with 8 to 30 C atoms and the Na, K, ammonium, Ca, Mg and Zn salts thereof.

The agents according to the invention preferably include the emulsifiers in quantities of 0.1-25 wt. %, in particular of 0.5-15 wt. %, relative to the total agent.

The compositions according to the invention may preferably include at least one nonionogenic emulsifier with an HLB value of 8 to 18. Nonionogenic emulsifiers with an HLB value of 10-15 may be more preferred according to the invention.

It has furthermore proven advantageous if, in addition to the polymer(s) from the group of cationic and/or amphoteric polymers, further polymers (G) are present in the agents according to the invention. In a preferred embodiment, the polymers are therefore added to the agents according to the invention, wherein both anionic and nonionic polymers have proven effective.

The anionic polymers (G2) are anionic polymers which comprise carboxylate and/or sulfonate groups. Examples of anionic monomers of which such polymers may consist are acrylic acid, methacrylic acid, crotonic acid, maleic anhydride and 2-acrylamido-2-methylpropanesulfonic acid. In this case, the acidic groups may be present wholly or in part as a sodium, potassium, ammonium, mono- or triethanolammonium salt. 2-Acrylamido-2-methylpropanesulfonic acid and acrylic acid are preferred monomers. Anionic polymers which have proven very particularly effective are those which include as sole or co-monomer 2-acrylamido-2-methylpropanesulfonic acid, wherein the sulfonic acid group may be present wholly or in part as a sodium, potassium, ammonium, mono- or triethanolammonium salt.

The homopolymer of 2-acrylamido-2-methylpropanesulfonic acid is more preferred, and is commercially obtainable for example under the name Rheothik® 11-80.

Within this embodiment it may be preferable to use copolymers of at least one anionic monomer and at least one nonionogenic monomer. With regard to anionic monomers, reference is made to the above-listed substances. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers including sulfonic acid groups. A more preferred anionic copolymer consists of 70 to 55 mol % acrylamide and 30 to 45 mol % 2-acrylamido-2-methylpropanesulfonic acid, wherein the sulfonic acid group is wholly or in part in the form of a sodium, potassium, ammonium, mono- or triethanolammonium salt. This copolymer may also be present in crosslinked form, wherein polyolefinically unsaturated compounds such as tetraallyloxyethane, allyl sucrose, allyl pentaerythritol and methylene bisacrylamide are preferably used as the crosslinking agents. Such a polymer is present in the commercial product Sepigel®305 from SEPPIC. Use of this compound, which includes in addition to the polymer component a hydrocarbon mixture (C₁₃-C₁₄ isoparaffin) and a nonionogenic emulsifier (Laureth-7), has proven particularly advantageous for the purposes of the teaching according to the invention.

The sodium acryloyldimethyl taurate copolymers distributed under the name Simulgel®600 as a compound with isohexadecane and polysorbate-80 have also proven particularly effective according to the invention.

Anionic homopolymers which are likewise preferred are uncrosslinked and crosslinked polyacrylic acids. In this case, allyl ethers of pentaerythritol, of sucrose and of propylene may be preferred crosslinking agents. Such compounds are commercially obtainable for example under the trademark Carbopol®.

Copolymers of maleic anhydride and methyl vinyl ether, in particular those comprising crosslinks, are also color-preserving polymers. A maleic acid-methyl vinyl ether copolymer crosslinked with 1,9-decadiene is commercially obtainable under the name Stabileze® QM.

In a further embodiment, the agents according to the invention may include nonionogenic polymers (G4).

Suitable nonionogenic polymers are for example:
vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, as are distributed for example under the tradenames Culminal® and Benecel® (AQUALON) and Natrosol® grades (Hercules).
starch and the derivatives thereof, in particular starch ethers, for example Structure® XL (National Starch), a multifunctional, salt-tolerant starch; shellac
polyvinylpyrrolidones, as are distributed for example under the tradename Luviskol® (BASF).
siloxanes. These siloxanes may be either water-soluble or water-insoluble. Both volatile and non-volatile siloxanes are suitable, wherein non-volatile siloxanes are taken to be those compounds whose boiling point at standard pressure is above 200° C. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes, which include amine and/or hydroxy groups.
glycosidically substituted silicones.

It is also possible according to the invention for the preparations to include a plurality of, in particular two different, identically charged polymers and/or in each case one ionic and one amphoteric and/or nonionic polymer.

The further polymers (G) are present in the agents according to the invention preferably in quantities of from 0.05 to 10 wt. %, relative to the total agent. Quantities of 0.1 to 5, in particular of 0.1 to 3 wt. %, are more preferred.

The present invention also provides a method for treating keratinic fibers, in which a hair treatment agent according to the invention is applied onto the keratinic fibers and, after a period of exposure of a few seconds up to 45 minutes, rinsed back out.

The above statements regarding the agents according to the invention apply mutatis mutandis with regard to preferred embodiments of the methods according to the invention.

The present invention also provides the use of hair treatment agents according to the invention
for conditioning keratin substances and/or
in order to improve volume, softness, gloss and/or combability and to facilitate styling of keratin substances and/or
in order to improve the persistence of the conditioning action on hair washing and/or
for improving wet and dry combability and/or
for improving gloss and/or
for improving the moisture balance of keratinic fibers and/or
for protecting keratinic fibers from oxidative damage and/or
for preventing keratinic fibers from becoming greasy again and/or
for increasing the washing resistance of dyed keratinic fibers.

The above statements regarding the agents according to the invention also apply mutatis mutandis with regard to further preferred embodiments of the uses according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition including, in a cosmetically acceptable medium,
(a) at least one thickener selected from the group consisting of:
(i.) associative thickeners;
(ii.) crosslinked homopolymers of acrylic acid;
(iii.) crosslinked copolymers of (meth)acrylic acid and alkyl(C₁₋₆) acrylate;
(iv.) nonionic homopolymers and copolymers which include monomers with ethylenically unsaturated bonds of the ester and/or amide type;
(v.) homopolymers of ammonium acrylate or copolymers of ammonium acrylate and acrylamide; and
(vi.) fatty acid amides; and
(b) at least one hydroxy-terminated organopolysiloxane of general formula (I), 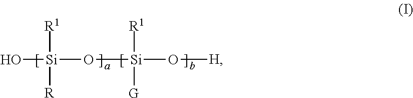
wherein
R means a monovalent unsubstituted or halo-substituted hydrocarbon residue with 1 to 20 carbon atoms,
R¹ means a monovalent unsubstituted or halo-substituted hydrocarbon residue with 1 to 20 carbon atoms, —OR⁴ or —OH,
R⁴ means an alkyl residue with 1 to 6 carbon atoms,
G means a group of general formula (II) 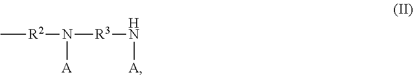
wherein
R², R³ mutually independently mean a divalent hydrocarbon residue with 1 to 6 carbon atoms, wherein non-adjacent —CH₂ units may be replaced by units which are selected from —C(═O)—, —O—, and —S—,
A means R⁵—C(═O)—,
R⁵ means an alkyl residue with 1 to 20 carbon atoms,
a means integral values of 100 to 1500, and
b means integral values of at least 1.

2. The cosmetic composition according to claim 1, wherein the R is selected from the group consisting of: methyl, ethyl, n-propyl, isopropyl, n-butyl, isobutyl, sec-butyl, tert-butyl, n-pentyl, and n-hexyl residues.

3. The cosmetic composition according to claim 1, wherein R is methyl.

4. The cosmetic composition according to claim 1, wherein residues R², R³ are selected from the group consisting of: ethylene, n-propylene, iso-butylene and n-butylene residues.

5. The cosmetic composition according to claim 1, wherein R⁵ is selected from the group consisting of: methyl, ethyl, n-propyl, isopropyl, n-butyl, isobutyl, sec-butyl, tert-butyl, n-pentyl, and n-hexyl residues.

6. The cosmetic composition according to claim 1, wherein R⁵ is methyl or ethyl.

5. The cosmetic composition according to claim 1, wherein the hydroxy-terminated organopolysiloxane(s) of general formula (I) constitutes 0.00001 to 10 wt. % of the composition.

6. The cosmetic composition according to claim 1, characterized in that, relative to the weight thereof, the cosmetic composition includes 0.00001 to 5 wt. % branched, ethoxylated tridecanol (INCI name: Trideceth-5) or α-iso-tridecyl-w-hydroxy polyglycol ether (INCI name: Trideceth-10) or mixtures thereof.

7. The cosmetic composition according to claim 1, wherein the weight-average molar mass of the hydroxy-terminated organopolysiloxane of general formula (I) is in the range from 2,000 to 1,000,000 gmol⁻¹.

8. The cosmetic composition according to claim 1, wherein the hydroxy-terminated organopolysiloxane of general formula (I) assumes the form of an oil-in-water emulsion in which the number-average size of the silicone particles in the emulsion is in the range from 3 to 500 nm.

9. The cosmetic composition according to claim 1, wherein the associative thickener is an associative polymer selected from the group consisting of
(i.) nonionic amphiphilic polymers which include at least one fatty chain and at least one hydrophilic unit;
(ii.) anionic amphiphilic polymers which include at least one hydrophilic unit and at least one unit with a fatty chain;
(iii.) cationic amphiphilic polymers which include at least one hydrophilic unit and at least one unit with a fatty chain; and
(iv.) amphoteric amphiphilic polymers which include at least one hydrophilic unit and at least one unit with a fatty chain,
and wherein the fatty chains have 10 to 30 carbon atoms.

10. The cosmetic composition according to claim 1, wherein the nonionic amphiphilic polymers which include at least one fatty chain and at least one hydrophilic unit are preferably selected from the group consisting of
(1) celluloses which are modified with groups which have at least one fatty chain;
(2) hydroxypropyl guar compounds which are modified with groups which have at least one fatty chain;
(3) urethane polyethers which include at least one fatty chain, such as alkyl or alkenyl groups with 10 to 30 carbon atoms;
(4) copolymers of vinylpyrrolidone and hydrophobic monomers with a fatty chain;
(5) copolymers of alkyl(C₁₋₆) methacrylates or alkyl(C₁₋₆) acrylates and amphiphilic monomers which include at least one fatty chain; and
(6) copolymers of hydrophilic methacrylates or acrylates and hydrophobic monomers which include at least one fatty chain.

11. The cosmetic composition according to claim 1, wherein the anionic amphiphilic polymers which include at least one hydrophilic unit and at least one unit with a fatty chain are selected from the group consisting of compounds which include at least one allyl ether unit with a fatty chain and at least one hydrophilic unit, compounds synthesized from an ethylenically unsaturated anionic monomer, compounds which include at least one hydrophilic unit of the type of an olefinically unsaturated carboxylic acid and at least one hydrophobic unit exclusively of the type of an alkyl(C₁₋₃₀) ester of an unsaturated carboxylic acid, and methacrylic acid/methyl acrylate/dimethyl-meta-isopropenylbenzyl isocyanate copolymers of an ethoxylated alcohol.

12. The cosmetic composition according to claim 1, wherein the cationic amphiphilic polymers are selected from the group consisting of quaternized cellulose derivatives and polyacrylates with aminated side chains.

13. The cosmetic composition according to claim 1, wherein the amphoteric amphiphilic polymers which include at least one fatty chain are selected from the group consisting of copolymers of methacrylamidopropyltrimethylammonium chloride/acrylic acid/alkyl(C₁₀₋₃₀) methacrylate.

14. The cosmetic composition according to claim 1, wherein the polysaccharides are selected from the group consisting of glucans, modified or unmodified starch compounds, amylose, amylopectin, glycogen, dextrans, celluloses and the derivatives thereof, mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carrageenins, agar-agar, glycosaminoglucans, gum arabic, tragacanth, gum ghatti, karaya gum, locust bean flour, galactomannans, xanthan gum and mixtures thereof.

15. The cosmetic composition according to claim 1, wherein the thickener(s) are included at a total quantity of 0.001 to 20 wt. %, relative to the total weight of the composition.

16. A method for treating keratinic fibers, characterized in that the hair treatment agent according to claim 1 is applied onto the keratinic fibers and, after a period of exposure of a few seconds up to 45 minutes, rinsed back out.
